(19) United States
(12) Patent Application Publication
(10) Pub. No.: US 2025/0312079 A1
(43) Pub. Date: Oct. 9, 2025

(54) SYSTEMS AND METHODS FOR PULSED FIELD ABLATION

(71) Applicant: KARDIUM INC., Burnaby (CA)

(72) Inventor: Daniel Martin REINDERS, Surrey (CA)

(21) Appl. No.: 19/095,132

(22) Filed: Mar. 31, 2025

Related U.S. Application Data
(60) Provisional application No. 63/573,565, filed on Apr. 3, 2024.

(51) Int. Cl. A61B 18/00

(52) U.S. Cl. CPC A61B 18/00; A61B 2018/0016; A61B 2018/00178; A61B 2018/00267; A61B 2018/00577; A61B 2018/00767

(57) ABSTRACT

A medical system may be configured at least to receive a user-selection of a first group of electrodes in a spatial distribution of electrodes; cause, at least in response to the user-selection of the first group of electrodes, a machine-selection of a second group of electrodes in the spatial distribution of electrodes, each electrode in the machine-selected second group of electrodes spaced, according to the spatial distribution, from an electrode in the user-selected first group of electrodes at least by a respective interposed electrode set in the spatial distribution of electrodes; and cause bipolar pulsed field tissue ablation between the user-selected first group of electrodes and the machine-selected second group of electrodes, while omitting at least any electrode of each respective interposed electrode set from causing bipolar pulsed field tissue ablation.

### CROSS-REFERENCE TO RELATED APPLICATION

This application claims the benefit of U.S. Provisional Application No. 63/573,565, filed Apr. 3, 2024, the entire disclosure of which is hereby incorporated herein by reference.

### TECHNICAL FIELD

Aspects of this disclosure generally are related to systems and methods for pulsed field ablation, such systems and methods applicable to medical systems.

### BACKGROUND

Cardiac surgery was initially undertaken using highly invasive open procedures. A sternotomy, which is a type of incision in the center of the chest that separates the sternum was typically employed to allow access to the heart. In the past several decades, more and more cardiac operations are performed using intravascular or percutaneous techniques, where access to inner organs or other tissue is gained via a catheter.

Intravascular or percutaneous surgeries benefit patients by reducing surgery risk, complications and recovery time. However, the use of intravascular or percutaneous technologies also raises some particular challenges. Medical devices used in intravascular or percutaneous surgery need to be deployed via catheter systems which significantly increase the complexity of the device structure. As well, doctors do not have direct visual contact with the medical devices once the devices are positioned within the body.

One example of where intravascular or percutaneous medical techniques have been employed is in the treatment of a heart disorder called atrial fibrillation. Atrial fibrillation is a disorder in which spurious electrical signals cause an irregular heartbeat. Atrial fibrillation has been treated with various methods including a technique known as “PV (pulmonary vein) isolation”. Research has shown that atrial fibrillation typically begins in the pulmonary veins or at the point where they attach to the left atrium. There are typically four major pulmonary veins, and some or all may be a focal point for activity that may cause atrial fibrillation. During this procedure, physicians create specific patterns of lesions in the heart to block various paths taken by the spurious electrical signals. The patterns of lesions may include a pattern of one or more lesions that encircle at least one of the pulmonary veins. Lesions were originally created using incisions, but are now typically created by ablating the tissue with various techniques including radiofrequency (“RF”) ablation, microwave ablation, laser ablation, and cryogenic ablation.

Recently, a new ablation modality known as pulsed field ablation (PFA) has gained significant popularity in the ablation of various tissue structures, for example, in cardiac ablation. PFA is an ablation method that employs high voltage pulse delivery in proximity to target tissue. The electric field applied by the high voltage pulses in PFA physiologically changes the tissue cells to which the energy is applied (e.g., puncturing or perforating the cell membrane to form various pores therein). If a relatively low field strength is established, the formed pores may close in time and cause the cells to maintain viability (e.g., a process sometimes referred to as reversible electroporation). If relatively greater field strength is established, then permanent, and sometimes larger, pores form in the tissue cells, the pores allowing loss of control of ion concentration gradients (both inward and outward) thereby resulting in cell death (e.g., in a process sometimes referred to as irreversible electroporation). In contrast to thermal ablation techniques such as RF ablation and cryogenic ablation, PFA ablation is considered to be “non-thermal” in nature since the resulting tissue cellular death or destruction is not primarily or substantially dependent on thermal processes.

PFA may employ high voltage pulse delivery in a monopolar mode (or bipolar mode) during the ablation process. Monopolar PFA generates an electric field (i.e., configured to cause tissue irreversible electroporation) between one or more transducers (e.g., electrodes) located in proximity to target tissue located within the body (e.g., within a bodily organ) and one or more neutral electrodes (i.e., sometimes referred to as an indifferent electrode) conventionally located on an external surface (e.g., a skin surface) of the body. Bipolar PFA generates an electric field (i.e., configured to cause tissue irreversible electroporation) between two or more transducers (e.g., electrodes) located in proximity to target tissue located within the body (e.g., within a bodily organ). Monopolar PFA typically generates an electric field (i.e., configured to cause tissue irreversible electroporation) that tends to extend (at a given field strength) more deeply into the target tissue as compared to bipolar PFA due to the return electrode being external to the body in which the internal treatment electrode is treating the target tissue, as compared to the bipolar PFA case where the treatment electrodes adjacent the target tissue being treated also provide internal return electrode(s). However, monopolar PFA is typically associated with a deeper zone of nerve/muscle stimulation which can create undesired muscle contractions. Bipolar PFA will typically lead to relatively lower muscle contraction effects, but will also generate weaker ablation effects that produce relatively shallower lesions.

Accordingly, the present inventor recognized that there is a need in the art for improved PFA device systems or control mechanisms thereof with improved capabilities that blend the benefits of monopolar PFA and bipolar PFA while mitigating their respective associated problems.

### SUMMARY

At least the above-discussed need is addressed and technical solutions are achieved in the art by various embodiments of the present invention. In some embodiments, a medical system includes an input-output device system communicatively connectable to each electrode in a spatial distribution of electrodes provided by a catheter, each electrode in the spatial distribution of electrodes configured to be contactable with a tissue surface of a bodily cavity; a memory device system storing a program; and a data processing device system communicatively connected to the input-output device system and the memory device system. In some embodiments, the data processing device system is configured at least by the program at least to: receive, via the input-output device system, a user-selection of a first group of electrodes in the spatial distribution of electrodes; cause, at least in response to the user-selection of the first group of electrodes, a machine-selection of a second group of electrodes in the spatial distribution of electrodes, the machine-selected second group of electrodes not including any electrode in the user-selected first group of electrodes, and the machine-selection selecting each electrode in the machine-selected second group of electrodes at least as a particular electrode in the spatial distribution of electrodes that is spaced, according to the spatial distribution, from an electrode in the user-selected first group of electrodes at least by a respective interposed electrode set in the spatial distribution of electrodes, each respective interposed electrode set including a particular number of particular electrodes equal to or exceeding a threshold number of one or more electrodes, each respective interposed electrode set not including (a) any electrode in the user-selected first group of electrodes, and not including (b) any electrode in the machine-selected second group of electrodes; and cause, via the input-output device system, particular activation of each electrode in the user-selected first group of electrodes and each electrode in the machine-selected second group of electrodes, the particular activation configured to cause bipolar pulsed field tissue ablation to occur while omitting at least any electrode of each respective interposed electrode set from undergoing any activation configured to cause bipolar pulsed field tissue ablation at least throughout the particular activation configured to cause bipolar pulsed field tissue ablation.

In some embodiments, the particular activation configured to cause bipolar pulsed field tissue ablation is configured to cause a voltage difference between each electrode in the user-selected first group of electrodes and each electrode in the machine-selected second group of electrodes, the voltage difference having a magnitude sufficient to cause bipolar pulsed field tissue ablation. In some embodiments, the particular activation configured to cause bipolar pulsed field tissue ablation includes concurrent activation of all electrodes in the user-selected first group of electrodes and all electrodes in the machine-selected second group of electrodes to cause bipolar pulsed field tissue ablation.

In some embodiments, the particular activation is configured to cause bipolar pulsed field tissue ablation while omitting, at least throughout the particular activation configured to cause bipolar pulsed field tissue ablation, (1) generation of any particular voltage difference having a magnitude sufficient to cause bipolar pulsed field tissue ablation between each electrode in the user-selected first group of electrodes and each other electrode in the user-selected first group of electrodes, (2) generation of any particular voltage difference having a magnitude sufficient to cause bipolar pulsed field tissue ablation between each electrode in the machine-selected second group of electrodes and each other electrode in the machine-selected second group of electrodes, or (1) and (2).

In some embodiments, the particular activation configured to cause bipolar pulsed field tissue ablation is configured to cause bipolar pulsed field tissue ablation to occur concurrently between each electrode in the user-selected first group of electrodes and all electrodes in the machine-selected second group of electrodes. In some embodiments, the particular activation configured to cause bipolar pulsed field tissue ablation includes concurrent activation of all electrodes in the user-selected first group of electrodes and all electrodes in the machine-selected second group of electrodes to cause the bipolar pulsed field tissue ablation.

In some embodiments, the particular activation configured to cause bipolar pulsed field tissue ablation is configured to cause bipolar pulsed field tissue ablation to occur concurrently between each electrode in the machine-selected second group of electrodes and all electrodes in the user-selected first group of electrodes. In some embodiments, the particular activation configured to cause bipolar pulsed field tissue ablation includes concurrent activation of all electrodes in the user-selected first group of electrodes and all electrodes in the machine-selected second group of electrodes to cause the bipolar pulsed field tissue ablation.

In some embodiments, the particular activation configured to cause bipolar pulsed field tissue ablation includes concurrent activation of all electrodes in the user-selected first group of electrodes and all electrodes in the machine-selected second group of electrodes to cause the bipolar pulsed field tissue ablation.

In some embodiments, the data processing device system is configured at least by the program at least to cause, at least in response to the machine-selection of the second group of electrodes and via the input-output device system, (i) each electrode in the user-selected first group of electrodes to be electrically connected to a first pole of a power supply system, and (ii) each electrode in the machine-selected second group of electrodes to be electrically connected to a second pole of the power supply system, the first pole and the second pole having opposite polarities at least throughout the particular activation configured to cause bipolar pulsed field tissue ablation. In some embodiments, the data processing device system is configured at least by the program at least to cause the power supply system to generate a pulsed voltage signal set between the first pole and the second pole to cause the particular activation configured to cause bipolar pulsed field tissue ablation. In some embodiments, the data processing device system is configured at least by the program at least to cause (i) and (ii) at least throughout the particular activation configured to cause bipolar pulsed field tissue ablation.

In some embodiments, the data processing device system is configured at least by the program at least to: receive, via the input-output device system, an electrode-to-tissue contact information set indicating a respective degree of electrode-to-tissue contact for each of at least some of the electrodes in the spatial distribution of electrodes; and cause the machine-selection of the second group of electrodes in the spatial distribution of electrodes based at least on an analysis of the electrode-to-tissue contact information set, such that the machine-selection selects the machine-selected second group of electrodes in a manner that each of at least one electrode in the machine-selected second group of electrodes is an electrode in the spatial distribution of electrodes that experiences a particular degree of electrode-to-tissue contact that is less than a respective degree of electrode-to-tissue contact experienced by each electrode in the user-selected first group of electrodes. In some embodiments, the at least one electrode in the machine-selected second group of electrodes that is an electrode in the spatial distribution of electrodes that experiences a particular degree of electrode-to-tissue contact that is less than a respective degree of electrode-to-tissue contact experienced by each electrode in the user-selected first group of electrodes includes a first electrode in the spatial distribution of electrodes that experiences no electrode-to-tissue contact.

In some embodiments, the data processing device system is configured at least by the program at least to: receive, via the input-output device system, an electrode-to-tissue contact information set indicating a respective degree of electrode-to-tissue contact for each of at least some of the electrodes in the spatial distribution of electrodes; and cause the machine-selection of the second group of electrodes in the spatial distribution of electrodes based at least on an analysis of the electrode-to-tissue contact information set, such that the machine-selection selects the machine-selected second group of electrodes in a manner that each electrode in the machine-selected second group of electrodes is an electrode in the spatial distribution of electrodes that experiences a particular degree of electrode-to-tissue contact that is less than a respective degree of electrode-to-tissue contact experienced by each electrode in the user-selected first group of electrodes. In some embodiments, the particular degree of electrode-to tissue contact is no electrode-to-tissue contact.

In some embodiments, the data processing device system is configured at least by the program at least to: receive, via the input-output device system, an electrode-to-tissue contact information set indicating a respective degree of electrode-to-tissue contact for each electrode in the spatial distribution of electrodes; and cause the machine-selection of the second group of electrodes in the spatial distribution of electrodes based at least on an analysis of the electrode-to-tissue contact information set, such that the machine-selection selects the machine-selected second group of electrodes in a manner that an average or median of the respective degrees of electrode-to-tissue contacts experienced by the electrodes in the machine-selected second group of electrodes is less than a respective one of an average or median of degrees of electrode-to-tissue contacts experienced by the electrodes in the user-selected first group of electrodes.

In some embodiments, each respective interposed electrode set in the spatial distribution of electrodes includes at least two electrodes adjacent one another according to the spatial distribution.

In some embodiments, each respective interposed electrode set in the spatial distribution of electrodes includes (i) an electrode that is adjacent a first electrode in the user-selected first group of electrodes according to the spatial distribution, and (ii) an electrode that is adjacent a second electrode in the machine-selected second group of electrodes according to the spatial distribution. In some embodiments, the electrode that is adjacent the first electrode in the user-selected first group of electrodes according to the spatial distribution is other than the electrode that is adjacent the second electrode in the machine-selected second group of electrodes according to the spatial distribution.

In some embodiments, a first electrode in the machine-selected second group of electrodes is spaced, according to the spatial distribution, from a particular electrode in the user-selected first group of electrodes by the respective interposed electrode set and at least a second electrode in the machine-selected second group of electrodes, the second electrode adjacent the first electrode according to the spatial distribution.

In some embodiments, a particular electrode in the machine-selected second group of electrodes is spaced, according to the spatial distribution, from a first electrode in the user-selected first group of electrodes by the respective interposed electrode set and at least a second electrode in the user-selected first group of electrodes, the second electrode adjacent the first electrode according to the spatial distribution.

In some embodiments, at least one electrode set of the respective interposed electrode sets in the spatial distribution of electrodes includes at least one electrode in the spatial distribution having a lower degree of electrode-to-tissue contact than any electrode in the user-selected first group of electrodes.

In some embodiments, at least one electrode set of the respective interposed electrode sets in the spatial distribution of electrodes includes at least one electrode having a lower degree of electrode-to-tissue contact than the associated electrode in the user-selected first group of electrodes.

In some embodiments, at least one electrode set of the respective interposed electrode sets in the spatial distribution of electrodes includes at least one electrode having no electrode-to-tissue contact.

In some embodiments, the memory device system stores data indicative of the threshold number of electrodes.

In some embodiments, the electrodes in the user-selected first group of electrodes are arranged in a ring-like arrangement according to the spatial distribution. In some embodiments, the electrodes in the machine-selected second group of electrodes are located outwardly of the ring-like arrangement according to the spatial distribution.

In some embodiments, the machine-selection of the machine-selected second group of electrodes is configured to cause the machine-selected second group of electrodes to have within 10% of the number of electrodes in the user-selected first group of electrodes.

In some embodiments, each electrode in the spatial distribution of electrodes includes a respective energy transmission surface configured to transmit tissue-ablative energy, and wherein the machine-selection of the machine-selected second group of electrodes is configured to cause the respective energy transmission surfaces of the machine-selected second group of electrodes to have a total combined surface area that is within 10% of a total combined surface area of the respective energy transmission surfaces of the user-selected first group of electrodes.

In some embodiments, the input-output device system is communicatively connected to each electrode in the spatial distribution of electrodes provided by the catheter.

In some embodiments, the tissue surface of the bodily cavity defines a volume of space within the bodily cavity, and wherein the machine-selection selects each electrode in the machine-selected second group of electrodes at least as a particular electrode in the spatial distribution of electrodes that is closer to an innermost region of the volume of space defined by the bodily cavity than any electrode in the user-selected first group of electrodes for inclusion in the machine-selected second group of electrodes. In some embodiments, the data processing device system is configured at least by the program at least to: receive, via the input-output device system, an electrode-to-tissue contact information set indicating a respective degree of electrode-to-tissue contact for each of at least some of the electrodes in the spatial distribution of electrodes; and cause the machine-selection of the second group of electrodes in the spatial distribution of electrodes based at least on an analysis of the electrode-to-tissue contact information set, such that the machine-selection selects the machine-selected second group of electrodes in a manner that each electrode in the machine-selected second group of electrodes is an electrode in the spatial distribution of electrodes that experiences a particular degree of electrode-to-tissue contact that is less than a respective degree of electrode-to-tissue contact experienced by each electrode in the user-selected first group of electrodes. In some embodiments, the particular degree of electrode-to tissue contact is no electrode-to-tissue contact.

In some embodiments, a medical system includes an input-output device system communicatively connectable to each electrode in a spatial distribution of electrodes provided by a catheter, each electrode in the spatial distribution of electrodes configured to be contactable with a tissue surface of a bodily cavity, the tissue surface of the bodily cavity defining a volume of space within the bodily cavity; a memory device system storing a program; and a data processing device system communicatively connected to the input-output device system and the memory device system. In some embodiments, the data processing device system is configured at least by the program at least to: receive, via the input-output device system, location information indicating a location of each of one or more portions of the catheter within the bodily cavity; receive, via the input-output device system, a user-selection of a first group of electrodes in the spatial distribution of electrodes; cause, at least in response to the user-selection of the first group of electrodes and based at least on an analysis of the received location information, a machine-selection of a second group of electrodes in the spatial distribution of electrodes, the machine-selected second group of electrodes not including any electrode in the user-selected first group of electrodes, and the machine-selection selecting, based at least on the analysis of the received location information, each electrode in the machine-selected second group of electrodes at least as a particular electrode in the spatial distribution of electrodes that is located closer to an innermost region within the volume of space defined by the bodily cavity than any electrode in the user-selected first group of electrodes; and cause, via the input-output device system, particular activation of each electrode in the user-selected first group of electrodes and each electrode in the machine-selected second group of electrodes.

In some embodiments, a medical system includes an input-output device system communicatively connectable to each electrode in a three-dimensional spatial distribution of electrodes provided by a catheter. In some embodiments, each electrode in the three-dimensional spatial distribution of electrodes includes a respective surface portion configured to be contactable with a tissue surface of a bodily cavity, the tissue surface of the bodily cavity defining a volume of space within the bodily cavity. In some embodiments, the respective surface portions of the electrodes face outwardly from a central region within the three-dimensional spatial distribution of electrodes. In some embodiments, the medical system includes a memory device system storing a program, and a data processing device system communicatively connected to the input-output device system and the memory device system. In some embodiments, the data processing device system is configured at least by the program at least to: receive, via the input-output device system, a user-selection of a first group of electrodes in the three-dimensional spatial distribution of electrodes; cause, at least in response to the user-selection of the first group of electrodes, a machine-selection of a second group of electrodes in the three-dimensional spatial distribution of electrodes, the machine-selected second group of electrodes not including any electrode in the user-selected first group of electrodes, and the respective surface portion of each of at least some electrodes in the machine-selected second group of electrodes facing toward an innermost region within the volume of space defined by the bodily cavity; and cause, via the input-output device system, particular activation of each electrode in the user-selected first group of electrodes and each electrode in the machine-selected second group of electrodes.

In some embodiments, a medical system includes an input-output device system communicatively connectable to each electrode in a three-dimensional spatial distribution of electrodes provided by a catheter, each electrode in the three-dimensional spatial distribution of electrodes including a respective surface portion configured to be contactable with a tissue surface of a bodily cavity. In some embodiments, the respective surface portions of the electrodes face outwardly from a central region within the three-dimensional spatial distribution of electrodes. In some embodiments, the medical system includes a memory device system storing a program, and a data processing device system communicatively connected to the input-output device system and the memory device system. In some embodiments, the data processing device system is configured at least by the program at least to: receive, via the input-output device system, a user-selection of a first group of electrodes in the three-dimensional spatial distribution of electrodes, the respective surface portion of each of at least some electrodes in the user-selected first group of electrodes facing toward a particular region of the tissue surface of the bodily cavity; cause, at least in response to the user-selection of the first group of electrodes, a machine-selection of a second group of electrodes in the three-dimensional spatial distribution of electrodes, the machine-selected second group of electrodes not including any electrode in the user-selected first group of electrodes, and the respective surface portion of each of at least some electrodes in the machine-selected second group of electrodes not facing toward the particular region of the bodily cavity; and cause, via the input-output device system, particular activation of each electrode in the user-selected first group of electrodes and each electrode in the machine-selected second group of electrodes.

Various embodiments of the present invention may include systems, devices, or machines that are or include combinations or subsets of any one or more of the systems, devices, or machines and associated features thereof summarized above or otherwise described herein (which should be deemed to include the figures).

Further, all or part of any one or more of the systems, devices, or machines summarized above or otherwise described herein or combinations or sub-combinations thereof may implement or execute all or part of any one or more of the processes or methods described herein or combinations or sub-combinations thereof.

For example, in some embodiments, a method is executed by a data processing device system according to a program stored by a communicatively connected memory device system, the data processing device system also communicatively connected to an input-output device system, the input-output device system communicatively connected to each electrode in a spatial distribution of electrodes provided by a catheter, each electrode in the spatial distribution of electrodes configured to be contactable with a tissue surface of a bodily cavity, the method including: receiving, via the input-output device system, a user-selection of a first group of electrodes in the spatial distribution of electrodes; causing, at least in response to the user-selection of the first group of electrodes, a machine-selection of a second group of electrodes in the spatial distribution of electrodes, the machine-selected second group of electrodes not including any electrode in the user-selected first group of electrodes, and the machine-selection selecting each electrode in the machine-selected second group of electrodes at least as a particular electrode in the spatial distribution of electrodes that is spaced, according to the spatial distribution, from an electrode in the user-selected first group of electrodes at least by a respective interposed electrode set in the spatial distribution of electrodes, each respective interposed electrode set including a particular number of particular electrodes equal to or exceeding a threshold number of one or more electrodes, each respective interposed electrode set not including (a) any electrode in the user-selected first group of electrodes, and not including (b) any electrode in the machine-selected second group of electrodes; and causing, via the input-output device system, particular activation of each electrode in the user-selected first group of electrodes and each electrode in the machine-selected second group of electrodes, the particular activation configured to cause bipolar pulsed field tissue ablation to occur while omitting at least any electrode of each respective interposed electrode set from undergoing any activation configured to cause bipolar pulsed field tissue ablation at least throughout the particular activation configured to cause bipolar pulsed field tissue ablation.

In some embodiments, a method is executed by a data processing device system according to a program stored by a communicatively connected memory device system, the data processing device system also communicatively connected to an input-output device system, the input-output device system communicatively connected to each electrode in a spatial distribution of electrodes provided by a catheter, each electrode in the spatial distribution of electrodes configured to be contactable with a tissue surface of a bodily cavity, the tissue surface of the bodily cavity defining a volume of space within the bodily cavity, and the method including: receiving, via the input-output device system, location information indicating a location of each of one or more portions of the catheter within the bodily cavity; receiving, via the input-output device system, a user-selection of a first group of electrodes in the spatial distribution of electrodes; causing, at least in response to the user-selection of the first group of electrodes and based at least on an analysis of the received location information, a machine-selection of a second group of electrodes in the spatial distribution of electrodes, the machine-selected second group of electrodes not including any electrode in the user-selected first group of electrodes, and the machine-selection selecting, based at least on the analysis of the received location information, each electrode in the machine-selected second group of electrodes at least as a particular electrode in the spatial distribution of electrodes that is located closer to an innermost region within the volume of space defined by the bodily cavity than any electrode in the user-selected first group of electrodes; and causing, via the input-output device system, particular activation of each electrode in the user-selected first group of electrodes and each electrode in the machine-selected second group of electrodes.

In some embodiments, a method is executed by a data processing device system according to a program stored by a communicatively connected memory device system, the data processing device system also communicatively connected to an input-output device system, the input-output device system communicatively connected to each electrode in a three-dimensional spatial distribution of electrodes provided by a catheter, each electrode in the three-dimensional spatial distribution of electrodes including a respective surface portion configured to be contactable with a tissue surface of a bodily cavity, the tissue surface of the bodily cavity defining a volume of space within the bodily cavity, wherein the respective surface portions of the electrodes face outwardly from a central region within the three-dimensional spatial distribution of electrodes, the method including: receiving, via the input-output device system, a user-selection of a first group of electrodes in the three-dimensional spatial distribution of electrodes; causing, at least in response to the user-selection of the first group of electrodes, a machine-selection of a second group of electrodes in the three-dimensional spatial distribution of electrodes, the machine-selected second group of electrodes not including any electrode in the user-selected first group of electrodes, and the respective surface portion of each of at least some electrodes in the machine-selected second group of electrodes facing toward an innermost region within the volume of space defined by the bodily cavity; and causing, via the input-output device system, particular activation of each electrode in the user-selected first group of electrodes and each electrode in the machine-selected second group of electrodes.

In some embodiments, a method is executed by a data processing device system according to a program stored by a communicatively connected memory device system, the data processing device system also communicatively connected to an input-output device system, the input-output device system communicatively connected to each electrode in a three-dimensional spatial distribution of electrodes provided by a catheter, each electrode in the three-dimensional spatial distribution of electrodes including a respective surface portion configured to be contactable with a tissue surface of a bodily cavity, wherein the respective surface portions of the electrodes face outwardly from a central region within the three-dimensional spatial distribution of electrodes, the method including: receiving, via the input-output device system, a user-selection of a first group of electrodes in the three-dimensional spatial distribution of electrodes, the respective surface portion of each of at least some electrodes in the user-selected first group of electrodes facing toward a particular region of the tissue surface of the bodily cavity; causing, at least in response to the user-selection of the first group of electrodes, a machine-selection of a second group of electrodes in the three-dimensional spatial distribution of electrodes, the machine-selected second group of electrodes not including any electrode in the user-selected first group of electrodes, and the respective surface portion of each of at least some electrodes in the machine-selected second group of electrodes not facing toward the particular region of the bodily cavity; and causing, via the input-output device system, particular activation of each electrode in the user-selected first group of electrodes and each electrode in the machine-selected second group of electrodes.

It should be noted that various embodiments of the present invention include variations of the methods or processes summarized above or otherwise described herein (which should be deemed to include the figures) and, accordingly, are not limited to the actions described or shown in the figures or their ordering, and not all actions shown or described are required according to various embodiments. According to various embodiments, such methods may include more or fewer actions and different orderings of actions. Any of the features of all or part of any one or more of the methods or processes summarized above or otherwise described herein may be combined with any of the other features of all or part of any one or more of the methods or processes summarized above or otherwise described herein.

In addition, a computer program product may be provided that includes program code portions for performing some or all of any one or more of the methods or processes and associated features thereof described herein, when the computer program product is executed by a computer or other computing device or device system. Such a computer program product may be stored on one or more computer-readable storage mediums, also referred to as one or more computer-readable data storage mediums or a computer-readable storage medium system.

For example, in some embodiments, one or more computer-readable storage mediums store a program executable by a data processing device system communicatively connected to an input-output device system, the input-output device system communicatively connectable to each electrode in a spatial distribution of electrodes provided by a catheter, each electrode in the spatial distribution of electrodes configured to be contactable with a tissue surface of a bodily cavity, the program including: reception instructions configured to cause reception, via the input-output device system, of a user-selection of a first group of electrodes in the spatial distribution of electrodes; machine-selection instructions configured to cause, at least in response to the user-selection of the first group of electrodes, a machine-selection of a second group of electrodes in the spatial distribution of electrodes, the machine-selected second group of electrodes not including any electrode in the user-selected first group of electrodes, and the machine-selection selecting each electrode in the machine-selected second group of electrodes at least as a particular electrode in the spatial distribution of electrodes that is spaced, according to the spatial distribution, from an electrode in the user-selected first group of electrodes at least by a respective interposed electrode set in the spatial distribution of electrodes, each respective interposed electrode set including a particular number of particular electrodes equal to or exceeding a threshold number of one or more electrodes, each respective interposed electrode set not including (a) any electrode in the user-selected first group of electrodes, and not including (b) any electrode in the machine-selected second group of electrodes; and activation instructions configured to cause, via the input-output device system, particular activation of each electrode in the user-selected first group of electrodes and each electrode in the machine-selected second group of electrodes, the particular activation configured to cause bipolar pulsed field tissue ablation to occur while omitting at least any electrode of each respective interposed electrode set from undergoing any activation configured to cause bipolar pulsed field tissue ablation at least throughout the particular activation configured to cause bipolar pulsed field tissue ablation.

In some embodiments, one or more computer-readable storage mediums store a program executable by a data processing device system communicatively connected to an input-output device system, the input-output device system communicatively connectable to each electrode in a spatial distribution of electrodes provided by a catheter, each electrode in the spatial distribution of electrodes configured to be contactable with a tissue surface of a bodily cavity, the tissue surface of the bodily cavity defining a volume of space within the bodily cavity, and the program including: first reception instructions configured to cause reception, via the input-output device system, of location information indicating a location of each of one or more portions of the catheter within the bodily cavity; second reception instructions configured to cause reception, via the input-output device system, of a user-selection of a first group of electrodes in the spatial distribution of electrodes; machine-selection instructions configured to cause, at least in response to the user-selection of the first group of electrodes and based at least on an analysis of the received location information, a machine-selection of a second group of electrodes in the spatial distribution of electrodes, the machine-selected second group of electrodes not including any electrode in the user-selected first group of electrodes, and the machine-selection selecting, based at least on the analysis of the received location information, each electrode in the machine-selected second group of electrodes at least as a particular electrode in the spatial distribution of electrodes that is located closer to an innermost region within the volume of space defined by the bodily cavity than any electrode in the user-selected first group of electrodes; and activation instructions configured to cause, via the input-output device system, particular activation of each electrode in the user-selected first group of electrodes and each electrode in the machine-selected second group of electrodes.

In some embodiments, one or more computer-readable storage mediums store a program executable by a data processing device system communicatively connected to an input-output device system, the input-output device system communicatively connectable to each electrode in a three-dimensional spatial distribution of electrodes provided by a catheter, each electrode in the three-dimensional spatial distribution of electrodes including a respective surface portion configured to be contactable with a tissue surface of a bodily cavity, the tissue surface of the bodily cavity defining a volume of space within the bodily cavity, wherein the respective surface portions of the electrodes face outwardly from a central region within the three-dimensional spatial distribution of electrodes, the program including: reception instructions configured to cause reception, via the input-output device system, of a user-selection of a first group of electrodes in the three-dimensional spatial distribution of electrodes; machine-selection instructions configured to cause, at least in response to the user-selection of the first group of electrodes, a machine-selection of a second group of electrodes in the three-dimensional spatial distribution of electrodes, the machine-selected second group of electrodes not including any electrode in the user-selected first group of electrodes, and the respective surface portion of each of at least some electrodes in the machine-selected second group of electrodes facing toward an innermost region within the volume of space defined by the bodily cavity; and activation instructions configured to cause, via the input-output device system, particular activation of each electrode in the user-selected first group of electrodes and each electrode in the machine-selected second group of electrodes.

In some embodiments, one or more computer-readable storage mediums store a program executable by a data processing device system communicatively connected to an input-output device system, the input-output device system communicatively connectable to each electrode in a three-dimensional spatial distribution of electrodes provided by a catheter, each electrode in the three-dimensional spatial distribution of electrodes including a respective surface portion configured to be contactable with a tissue surface of a bodily cavity, wherein the respective surface portions of the electrodes face outwardly from a central region within the three-dimensional spatial distribution of electrodes, the program including: reception instructions configured to cause reception, via the input-output device system, of a user-selection of a first group of electrodes in the three-dimensional spatial distribution of electrodes, the respective surface portion of each of at least some electrodes in the user-selected first group of electrodes facing toward a particular region of the tissue surface of the bodily cavity; machine-selection instructions configured to cause, at least in response to the user-selection of the first group of electrodes, a machine-selection of a second group of electrodes in the three-dimensional spatial distribution of electrodes, the machine-selected second group of electrodes not including any electrode in the user-selected first group of electrodes, and the respective surface portion of each of at least some electrodes in the machine-selected second group of electrodes not facing toward the particular region of the bodily cavity; and activation instructions configured to cause, via the input-output device system, particular activation of each electrode in the user-selected first group of electrodes and each electrode in the machine-selected second group of electrodes.

In some embodiments, each of any of one or more or all of the computer-readable storage mediums or medium systems (also referred to as processor-accessible memory device systems) described herein is a non-transitory computer-readable (or processor-accessible) data storage medium or medium system (or memory device system) including or consisting of one or more non-transitory computer-readable (or processor-accessible) storage mediums (or memory devices) storing the respective program(s) which may configure a data processing device system to execute some or all of any of one or more of the methods or processes described herein.

Further, any of all or part of one or more of the methods or processes and associated features thereof discussed herein may be implemented or executed on or by all or part of a device system, apparatus, or machine, such as all or a part of any of one or more of the systems, apparatuses, or machines described herein or a combination or sub-combination thereof.

### BRIEF DESCRIPTION OF THE DRAWINGS

It is to be understood that the attached drawings are for purposes of illustrating aspects of various embodiments and may include elements that are not to scale.

FIG. 1 includes a schematic representation of a transducer-activation system according to various example embodiments, the transducer-activation system including a data processing device system, an input-output device system, and a memory device system.

FIG. 2 includes a cutaway diagram of a heart showing a transducer-based device percutaneously placed in a left atrium of the heart, according to various example embodiments.

FIG. 3A includes a partially schematic representation of a medical system according to various example embodiments, the medical system including a data processing device system, an input-output device system, a memory device system, and a transducer-based device including a plurality of transducers and an expandable structure shown in a delivery or unexpanded configuration.

FIG. 3B includes the representation of the medical system of FIG. 3A with the expandable structure shown in a deployed or expanded configuration as viewed from a first viewing direction, according to some embodiments.

FIG. 3C includes the representation of the medical system of FIG. 3B with the expandable structure shown in a deployed or expanded configuration as viewed from a second viewing direction, according to some embodiments.

FIG. 4 includes a schematic representation of a transducer-based device that includes a flexible circuit structure, according to various example embodiments.

FIG. 5 includes a block diagram of methods for selecting electrodes for activation in a pulsed field tissue ablation procedure, according to some embodiments.

FIGS. 6A and 6B include representations of a transducer-based device percutaneously placed in a left atrium of a heart, with some electrodes shown with various patterns indicating user-selected and machine-selected electrodes, according to some embodiments.

FIGS. 7A, 7B, and 7C include models of electric fields generated by electrodes having a 1 mm separation (FIG. 7A), a 10 mm separation (FIG. 7B), and by a single electrode (FIG. 7C).

FIG. 7D includes a graph showing how a voltage gradient for a same electrode voltage varies with the radial distance from an electrode in a direction of lesion depth when the electrode is in contact with tissue, the graph generated from data employed to produces FIGS. 7A, 7B, and 7C.

FIG. 8 includes a block diagram of methods for selecting electrodes for activation in a pulsed field tissue ablation procedure, the selecting based at least on an analysis of location information, according to some embodiments.

### DETAILED DESCRIPTION

At least the above-discussed need is addressed, and technical solutions are achieved by various embodiments of the present invention. In some embodiments, an ablation device system is configured to perform a ‘pseudo-monopolar’ ablation with electrodes on a structure (such as a catheter) inside a bodily cavity, where the electrodes are sufficiently far apart to blend the benefits of a monopolar ablation (e.g., generating a relatively deep tissue lesion) and the benefits of bipolar ablation (e.g., relatively lower muscle contraction effects). For instance, in some embodiments, the inventive ‘pseudo-monopolar’ ablation may be implemented by providing bipolar pulsed field ablation between two electrodes (or two electrode sets) sufficiently apart within the bodily cavity, such that the ablation behaves at one of the electrodes (or electrode sets) in a manner similar to monopolar pulsed field ablation. The inventive ‘pseudo-monopolar’ also allows for the removal of an external (outside the patient body) indifferent electrode, which conventionally has been used to perform monopolar ablations using a first electrode in the bodily cavity performing the tissue ablation with the external surface indifferent electrode acting as a return for the energy delivered by the first electrode.

In some embodiments, the process of implementing the inventive ‘pseudo-monopolar’ ablation is made more efficient by allowing a user to select, via a user-interface, a first group of electrodes, and then, a second group of electrodes is machine-selected, the machine-selected second group of electrodes separated from the user-selected first group of electrodes by an interposed electrode set, for instance, in some contexts and embodiments in which all of the electrodes are in a spatial distribution about a structure of a catheter. A bipolar pulsed field ablation may then be configured to be performed between the first and second groups of electrodes, where the interposed electrode set can ensure, e.g., that the first and second groups of electrodes are sufficiently apart to produce the blended benefits described above (and described otherwise herein) of the inventive ‘pseudo-monopolar’ ablation.

It should be noted that various embodiments of the invention are not limited to these features and benefits, which are referred to for purposes of illustration only, and additional and alternative features and benefits will become apparent from the following description in conjunction with reference to the figures.

In this regard, in the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments. In one embodiment, all references to “some embodiments” may refer to the same single embodiment.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects. In some embodiments, the word “subset” is intended to mean a set having the same or fewer elements of those present in the subset's parent or superset. In other embodiments, the word “subset” is intended to mean a set having fewer elements of those present in the subset's parent or superset. In this regard, when the word “subset” is used, some embodiments of the present invention utilize the meaning that “subset” has the same or fewer elements of those present in the subset's parent or superset, and other embodiments of the present invention utilize the meaning that “subset” has fewer elements of those present in the subset's parent or superset.

Further, the phrase “at least” is or may be used herein at times merely to emphasize the possibility that other elements may exist besides those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments, and the word “system” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers or is a necessary precondition for the event A, according to various embodiments.

In some embodiments, the term “adjacent”, the term “proximate”, and the like refer at least to a sufficient closeness between the objects or events defined as adjacent, proximate, or the like, to allow the objects or events to interact in a designated way. For example, in the case of physical objects, if object A performs an action on an adjacent or proximate object B, objects A and B would have at least a sufficient closeness to allow object A to perform the action on object B. In this regard, some actions may require contact between the associated objects, such that if object A performs such an action on an adjacent or proximate object B, objects A and B would be in contact, for example, in some instances or embodiments where object A needs to be in contact with object B to successfully perform the action. In some embodiments, the word “adjacent”, the word “proximate”, and the like additionally or alternatively refer to objects or events that do not have another substantially similar object or event between them. For example, object or event A and object or event B could be considered adjacent or proximate (e.g., physically or temporally) if they are immediately next to each other (with no other object or event between them) or are not immediately next to each other but no other object or event that is substantially similar to object or event A, object or event B, or both objects or events A and B, depending on the embodiment, is between them. In the context of electrodes, discussed at various points in this description, two electrodes may be considered adjacent, in some embodiments, when the two electrodes have no other electrodes between them that is the same in function to any of the two electrodes (e.g., the electrodes are tissue ablation electrodes). In some embodiments, the term “adjacent”, the term “proximate”, and the like additionally or alternatively refer to at least a sufficient closeness between the objects or events defined as adjacent, proximate, and the like, the sufficient closeness being within a range that does not place any one or more of the objects or events into a different or dissimilar region or time period, or does not change an intended function of any one or more of the objects or events or of an encompassing object or event that includes a set of the objects or events. Different embodiments of the present invention adopt different ones or combinations of the above definitions. Of course, however, the word “adjacent”, the word “proximate”, and the like are not limited to any of the above example definitions, according to some embodiments. In addition, the word “adjacent” and the word “proximate” do not have the same definition, according to some embodiments.

The phrase “pulsed field ablation” (“PFA”) as used in this disclosure refers, in some embodiments, to an ablation method that employs high voltage pulse delivery in a monopolar or bipolar fashion in proximity to target tissue. In some embodiments, each high voltage pulse may be referred to as a discrete energy application. In some embodiments, a grouped plurality of high voltages pulses (e.g., a train or packet of pulses) may be referred to as a discrete energy application. Each high voltage pulse can be a monophasic pulse including a single polarity, or a biphasic pulse including a first component having a first particular polarity and a second component having a second particular polarity opposite the first particular polarity. In some embodiments, the second component of the biphasic pulse follows immediately after the first component of the biphasic pulse. In some embodiments, the first and second components of the biphasic pulse are temporally separated by a relatively short time interval (e.g., an inter-phase delay). In some embodiments, successive monophasic or biphasic pulses are separated by a period of time referred to as an inter-pulse delay. In some embodiments, the duration of the inter-pulse delay may be greater than the duration of each of the monophasic or biphasic pulses. In some embodiments, each high voltage pulse may include a multiphasic pulse, such as a triphasic pulse, that includes a first component having a first particular polarity, a second component having a second particular polarity opposite the first particular polarity, and a third component having a third particular polarity that is the same as the first particular polarity.

The word “proximal”, in the context of a proximal portion, proximal location, and the like of a medical device, includes, for example, the portion, location, and the like, being or being configured to be further away from a patient or portion of or region within a patient (e.g., a bodily cavity) intended to be treated or assessed by the medical device, as compared to a distal portion, location, and the like of the medical device, according to some embodiments. In some embodiments, the word “proximal”, in the context of a proximal portion, proximal location, and the like of a medical device, includes, for example, the portion, location, and the like, being or being configured to be delivered (e.g., percutaneously or intravascularly) toward a patient or portion of or region within a patient (e.g., a bodily cavity) intended to be treated or assessed by the medical device, after or behind a distal portion, location, and the like of the medical device.

On the other hand, the word “distal”, in the context of a distal portion, distal location, and the like of a medical device, includes, for example, the portion, location, and the like, being or being configured to be closer to a patient or portion of or region within a patient (e.g., a bodily cavity) intended to be treated or assessed by the medical device, as compared to a proximal portion, location, and the like of the medical device, according to some embodiments. In some embodiments, the word “distal”, in the context of a distal portion, distal location, and the like of a medical device, includes, for example, the portion, location, and the like, being or being configured to be delivered (e.g., percutaneously or intravascularly) toward a patient or portion of or region within a patient (e.g., a bodily cavity) intended to be treated or assessed by the medical device, before or ahead of a proximal portion, location, and the like of the medical device.

According to some embodiments, the word “fluid” as used in this disclosure should be understood to include any fluid that can be contained within a bodily cavity or can flow into or out of, or both into and out of a bodily cavity via one or more bodily openings positioned in fluid communication with the bodily cavity. In the case of cardiac applications, fluid such as blood will flow into and out of various intra-cardiac cavities (e.g., a left atrium or right atrium).

According to some embodiments, the words “bodily opening” as used in this disclosure should be understood to include a naturally occurring bodily opening or channel or lumen; a bodily opening or channel or lumen formed by an instrument or tool using techniques that can include, but are not limited to, mechanical, thermal, electrical, chemical, and exposure or illumination techniques; a bodily opening or channel or lumen formed by trauma to a body; or various combinations of one or more of the above. Various elements having respective openings, lumens, or channels and positioned within the bodily opening (e.g., a catheter sheath) may be present in various embodiments. These elements may provide a passageway through a bodily opening for various devices employed in various embodiments.

The words “bodily cavity” as used in this disclosure should be understood to mean a cavity in a body, in some embodiments. The bodily cavity may be a cavity or chamber provided in a bodily organ (e.g., an intracardiac cavity of a heart). The bodily cavity may be provided by a bodily vessel. The bodily cavity may be at least partially surrounded or enclosed by a tissue surface. The tissue surface of the bodily cavity may be said to define the bodily cavity.

The word “tissue” as used in this disclosure should be understood to include, for example, any surface-forming tissue that is used to form a surface of a body or a surface within a bodily cavity, a surface of an anatomical feature or a surface of a feature associated with a bodily opening positioned in fluid communication with the bodily cavity. The tissue may include, for example, part or all of a tissue wall or membrane that defines a surface of the bodily cavity. In this regard, the tissue may form an interior surface of the cavity that surrounds a fluid within the cavity. In the case of cardiac applications, tissue may include, for example, tissue used to form an interior surface of an intra-cardiac cavity such as a left atrium or right atrium. In some embodiments, tissue is non-excised tissue. In some embodiments, the word “tissue” may refer to a tissue having fluidic properties (e.g., blood) and may be referred to as fluidic tissue.

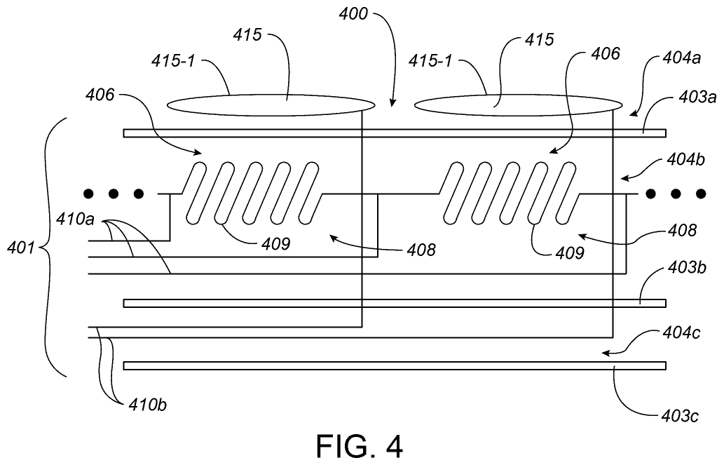

According to some embodiments, the word “transducer” as used in this disclosure should be interpreted broadly as any device configured to transmit or deliver energy; distinguish between fluid and tissue; sense temperature; generate heat; ablate tissue; sense, sample, or measure electrical activity of a tissue surface (e.g., sense, sample, or measure intracardiac electrograms, or sense, sample, or measure intracardiac voltage data); stimulate tissue; provide location information (e.g., in conjunction with a navigation system); or any combination thereof. A transducer may convert input energy of one form into output energy of another form. Without limitation, a transducer may include, for example, an electrode that functions as, or as part of, a sensing device included in the transducer, an energy delivery device included in the transducer, or both a sensing device and an energy delivery device included in the transducer. A transducer may be constructed from several parts, which may be discrete components or may be integrally formed. In this regard, although transducers, electrodes, or both transducers and electrodes are referenced with respect to various embodiments, it is understood that other transducers or transducer elements may be employed in other embodiments. It is understood that a reference to a particular transducer in various embodiments may also imply a reference to an electrode, as an electrode may be part of the transducer as shown, e.g., at least with FIG. 4 discussed below.

The term “activation” as used in this disclosure, according to some embodiments, should be interpreted broadly as making active a particular function as related to various transducers such as those disclosed herein, for example. Particular functions can include, but are not limited to, tissue ablation (e.g., PFA); sensing, sampling, or measuring electrophysiological activity (e.g., sensing, sampling, or measuring intracardiac electrogram information, or sensing, sampling, or measuring intracardiac voltage data); sensing, sampling, or measuring temperature; and sensing, sampling, or measuring electrical characteristics (e.g., tissue impedance or tissue conductivity). For example, in some embodiments, activation of a tissue ablation function of a particular transducer or electrode is initiated by causing energy sufficient for tissue ablation to be delivered to the particular transducer from an energy source device system (also known as a power supply system in some embodiments). In some embodiments, activation of a tissue ablation function of a particular transducer or electrode is initiated by causing energy sufficient for tissue ablation to be delivered by the particular transducer or electrode. Alternatively, in some embodiments, the activation can be deemed to be initiated when the particular transducer or particular electrode causes tissue that is to be ablated to exhibit tissue-ablative damage. In some embodiments, the activation can last for a duration concluding when the ablation function is no longer active, such as when energy sufficient for the tissue ablation is no longer delivered or provided to, or transmitted by, the particular transducer or particular electrode. Alternatively, in some embodiments, the activation period can be deemed to be concluded when the tissue that is being ablated no longer accrues tissue-ablative damage, which may be due to a reduction or cessation of the energy provided or transmitted by the energy source device system or delivered by the particular transducer or electrode. In some contexts and embodiments, however, the word “activation” may merely refer to the initiation of the activating of a particular function, as opposed to referring to both the initiation of the activating of the particular function and the subsequent duration in which the particular function is active. In these contexts, the phrase or a phrase similar to “activation initiation” may be used. For example, in some embodiments, activation initiation may cause initiation of a delivery of energy (e.g., energy sufficient for tissue ablation) from a particular transducer or electrode.

Some embodiments of the present invention may be implemented at least in part by a data processing device system or a controller system configured by a software program. Such a program may equivalently be implemented as multiple programs, and some or all of such software program(s) may be equivalently constructed in hardware. Reference to “a program” should be interpreted to include one or more programs.

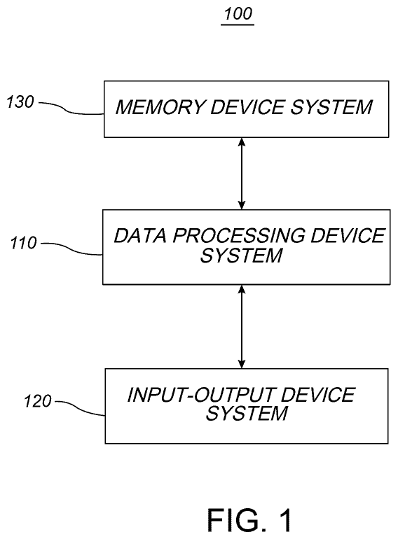

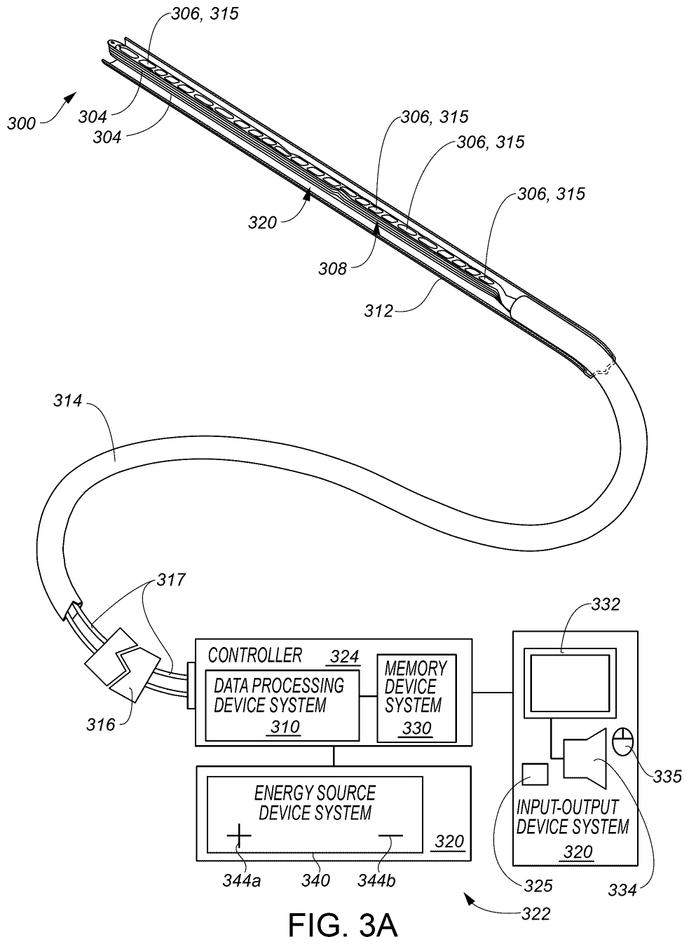

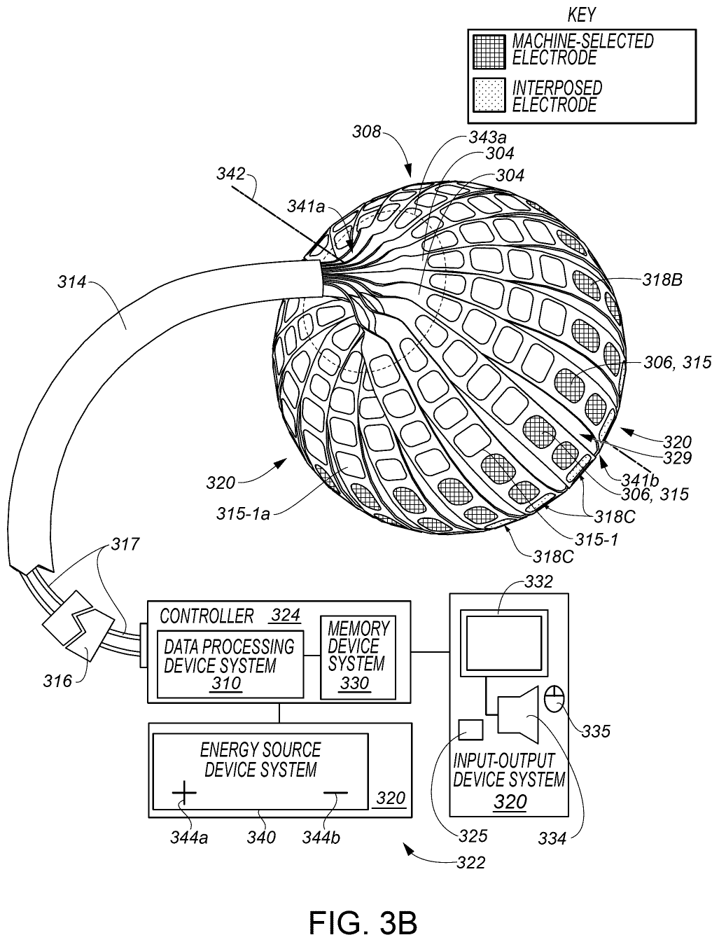

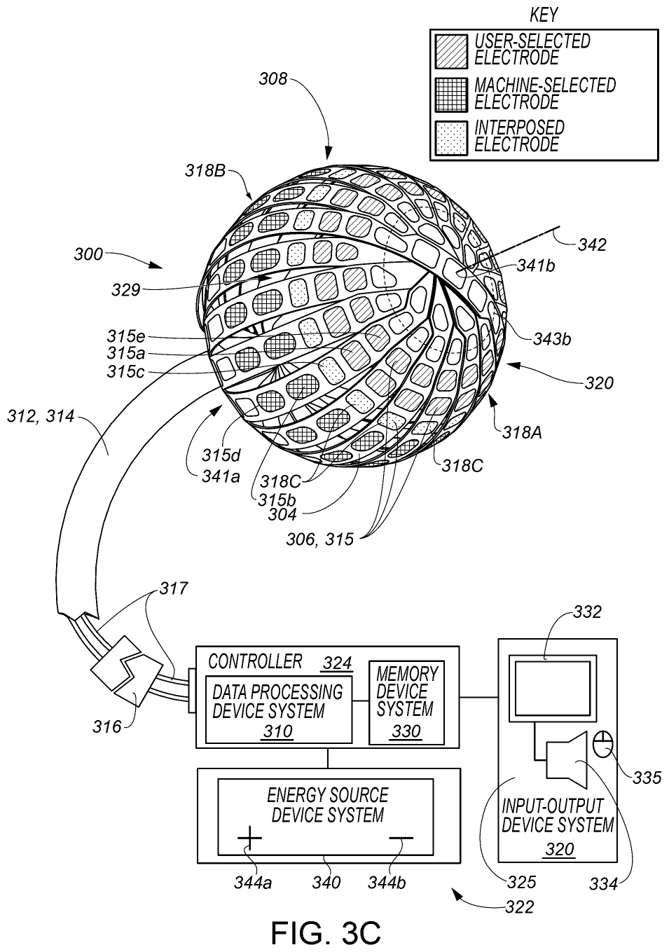

According to some embodiments, the term “program” in this disclosure should be interpreted to include one or more programs including a set of instructions or modules that may be executed by one or more components in a system, such as a controller system or data processing device system, in order to cause or configure the system to perform one or more operations. The set of instructions or modules may be stored by any kind of memory device, such as those described subsequently with respect to the memory device system 130, 330, or both, shown in FIGS. 1 and 3, respectively. In addition, this disclosure may describe or similarly describe that the instructions or modules of a program are configured to cause the performance of an action. The phrase “configured to” in this context is intended to include, for example, at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). In some instances, this disclosure may describe that the instructions or modules of a program perform an action. Such descriptions should be deemed to be equivalent to describing that the instructions or modules are configured to cause the performance of the action. The term “module” may be defined as a set of instructions. The term “program” and the term “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information, signals, or data or the like includes modifications to that information, signals, or data. For example, “data X” may be encrypted for transmission, and a reference to “data X” is intended to include both its encrypted and unencrypted forms, unless otherwise required or indicated by context. For another example, “image information Y” may undergo a noise filtering process, and a reference to “image information Y” is intended to include both the pre-processed form and the noise-filtered form, unless otherwise required or indicated by context.

In other words, both the pre-processed form and the noise-filtered form are considered to be “image information Y”, unless otherwise required or indicated by context. In order to stress this point, the phrase “or a derivative thereof” or the like may be used herein. Continuing the preceding example, the phrase “image information Y or a derivative thereof” refers to both the pre-processed form and the noise-filtered form of “image information Y”, unless otherwise required or indicated by context, with the noise-filtered form potentially being considered a derivative of “image information Y”. However, non-usage of the phrase “or a derivative thereof” or the like nonetheless includes derivatives or modifications of information or data unless otherwise explicitly noted or required by context.

In some embodiments, the phrase “graphical representation” used herein is intended to include a visual representation presented via a display device system and may include computer-generated text, graphics, animations, or one or more combinations thereof, which may include one or more visual representations originally generated, at least in part, by an image-capture device, such as computerized tomography (“CT”) scan images, magnetic resonance imaging (“MRI”) images, or images created from a navigation system (e.g., electropotential navigation system or an electro-magnetic navigation system), according to some embodiments. The graphical representation may include various entities depicted in a three-dimensional manner, in some embodiments. The graphical representation may include various entities depicted in a two-dimensional manner that are mapped from a three-dimensional space into a two-dimensional coordinate system, in some embodiments.

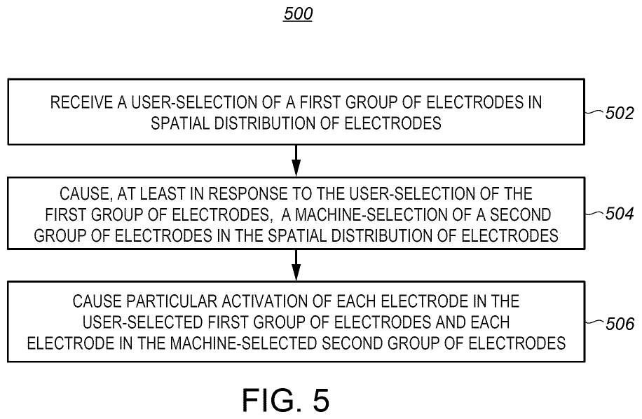

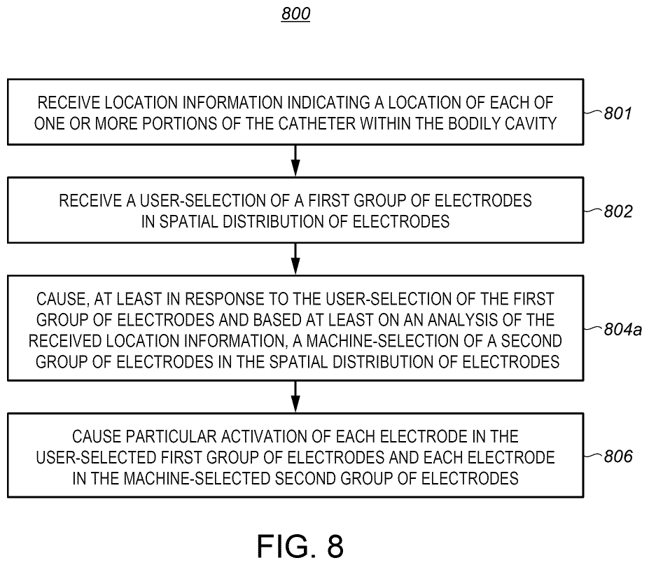

Example methods are described herein with respect to FIGS. 5 and 8. Each of FIGS. 5 and 8 includes blocks associated with actions, computer-executable instructions of one or more programs, or both actions and computer-executable instructions, according to various embodiments. It should be noted that the respective instructions associated with any such blocks therein need not be separate instructions and may be combined with other instructions to form a combined instruction set. The same set of instructions may be associated with more than one block. In this regard, the block arrangement shown in FIGS. 5 and 8 is not limited to an actual structure of any program or set of instructions or required ordering of method tasks, and such method figure, according to some embodiments, merely illustrates the tasks that instructions are configured to perform, for example, upon execution by a data processing device system in conjunction with interactions with one or more other devices or device systems.

FIG. 1 schematically illustrates a portion of a transducer-activation system or controller system thereof 100 that may be employed to at least select, control, activate, or monitor a function or activation of a number of electrodes or transducers (e.g., ablation transducers configured to cause thermal ablation or ablation transducers configured to cause PFA), according to some embodiments. The system 100 includes a data processing device system 110, an input-output device system 120, and a processor-accessible memory device system 130. The processor-accessible memory device system 130 and the input-output device system 120 are communicatively connected to the data processing device system 110. According to some embodiments, various components such as data processing device system 110, input-output device system 120, and processor-accessible memory device system 130 form at least part of a controller system (e.g., controller system 324 shown in FIG. 3).

The data processing device system 110 includes one or more data processing devices that implement or execute, in conjunction with other devices, such as those in the system 100, various methods and functions described herein, including those described with respect to methods exemplified in FIGS. 5 and 8. Each of the phrases “data processing device”, “data processor”, “processor”, “controller”, “computing device”, “computer” and the like is intended to include any data or information processing device, such as a central processing unit (CPU), a control circuit, a desktop computer, a laptop computer, a mainframe computer, a tablet computer, a personal digital assistant, a cellular or smart phone, and any other device for processing data, managing data, or handling data, whether implemented with electrical, magnetic, optical, or quantum components, or otherwise.

The memory device system 130 includes one or more processor-accessible memory devices configured to store one or more programs and information, including the program(s) and information needed to execute the methods or functions described herein, including those described with respect to FIGS. 5 and 8. The memory device system 130 may be a distributed processor-accessible memory device system including multiple processor-accessible memory devices communicatively connected to the data processing device system 110 via a plurality of computers and/or devices. However, the memory device system 130 need not be a distributed processor-accessible memory system and, consequently, may include one or more processor-accessible memory devices located within a single data processing device or housing.

Each of the phrases “processor-accessible memory” and “processor-accessible memory device” and the like is intended to include any processor-accessible data storage device or medium, whether volatile or nonvolatile, electronic, magnetic, optical, or quantum, or otherwise, including but not limited to, registers, hard disk drives, Compact Discs, DVDs, flash memories, ROMs, and RAMs. In some embodiments, each of the phrases “processor-accessible memory” and “processor-accessible memory device” is intended to include or be a processor-accessible (or computer-readable) data storage medium. In some embodiments, each of the phrases “processor-accessible memory” and “processor-accessible memory device” may include or may be a non-transitory processor-accessible (or computer-readable) data storage medium. In some embodiments, the processor-accessible memory device system 130 may include or may be a non-transitory processor-accessible (or computer-readable) data storage medium system. In some embodiments, the memory device system 130 may include or may be a non-transitory processor-accessible (or computer-readable) storage medium system or data storage medium system including or consisting of one or more non-transitory processor-accessible (or computer-readable) storage or data storage mediums.

The phrase “communicatively connected” is intended to include any type of connection, whether wired or wireless, between devices, data processors, or programs between which data may be communicated. Further, the phrase “communicatively connected” is intended to include a connection between devices or programs within a single data processor or computer, a connection between devices or programs located in different data processors or computers, and a connection between devices not located in data processors or computers at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing device system 110 or the memory device system 130, for example, depending upon the contents of the input-output device system 120. Further still, the data processing device system 110, the input-output device system 120, and the memory device system 130 may be located entirely within the same device or housing or may be separately located, but communicatively connected, among different devices or housings. In at least the case where the data processing device system 110, the input-output device system 120, and the memory device system 130 are located within the same device, the system 100 of FIG. 1 may be implemented by a single application-specific integrated circuit (ASIC), field programmable gate array (FPGA), system on chip (SOC), or other type of integrated circuit, in some embodiments. In this regard, in some embodiments, the processor-accessible memory device system 130 may be considered to be integrated with the data processing device system 110, such that circuitry or hardware may itself be encoded with the equivalent of computer-executable program instructions described herein to execute the methods and actions described herein, including those described with respect to the methods 500 and 800 of FIGS. 5 and 8. Such integration may be considered a type of communicative connection between the data processing device system 110 and the processor-accessible memory device system 130. Such integration may also be considered control circuitry or a controller that combines at least some functionality of the processor-accessible memory device system 130 and the data processing device system 110, such that the control circuitry or controller is communicatively connected to the input-output device system 120 and is configured (e.g., via circuitry or hardware encoded with the equivalent of computer-executable program instructions) at least to perform the methods and actions described herein, including those described with respect to the methods 500 and 800 of FIGS. 5 and 8.

The input-output device system 120 may include a mouse, a keyboard, a touch screen, another computer, a processor-accessible memory device system, a network-interface card or network-interface circuitry, or any device or combination of devices from which a desired selection, desired information, instructions, or any other data is input to the data processing device system 110. The input-output device system 120 may include any suitable interface for receiving information, instructions or any data from other devices and systems described in various ones of the embodiments. In this regard, the input-output device system 120 may include various ones of other systems described in various embodiments. For example, the input-output device system 120 may include at least a portion of a transducer-based device (e.g., a catheter, or portion thereof) that includes a spatial distribution of electrodes. The phrase “transducer-based device” or “transducer-based device system” is intended to include one or more physical systems that include various transducers (e.g., electrodes).

The input-output device system 120 also may include an image generating device system, a display device system, a speaker or audio output device system, a computer, a processor-accessible memory device system, a network-interface card or network-interface circuitry, or any device or combination of devices to which information, instructions, or any other data is output by the data processing device system 110. In this regard, the input-output device system 120 may include various other devices or systems described in various embodiments. The input-output device system 120 may include any suitable interface for outputting information, instructions, or data to other devices and systems described in various ones of the embodiments. If the input-output device system 120 includes a processor-accessible memory device, such memory device may, or may not, form part, or all, of the memory device system 130. The input-output device system 120 may include any suitable interface for outputting information, instructions, or data to other devices and systems described in various ones of the embodiments. In some embodiments, the input-output device system 120 may include a transducer-based device, as discussed above, and in some embodiments, the transducer-based device may act as a device or device system that provides information to, receives instructions or energy from, or both provides information to and receives instructions or energy from the data processing device system 110. In this regard, the input-output device system 120 may include various devices or systems described in various embodiments.

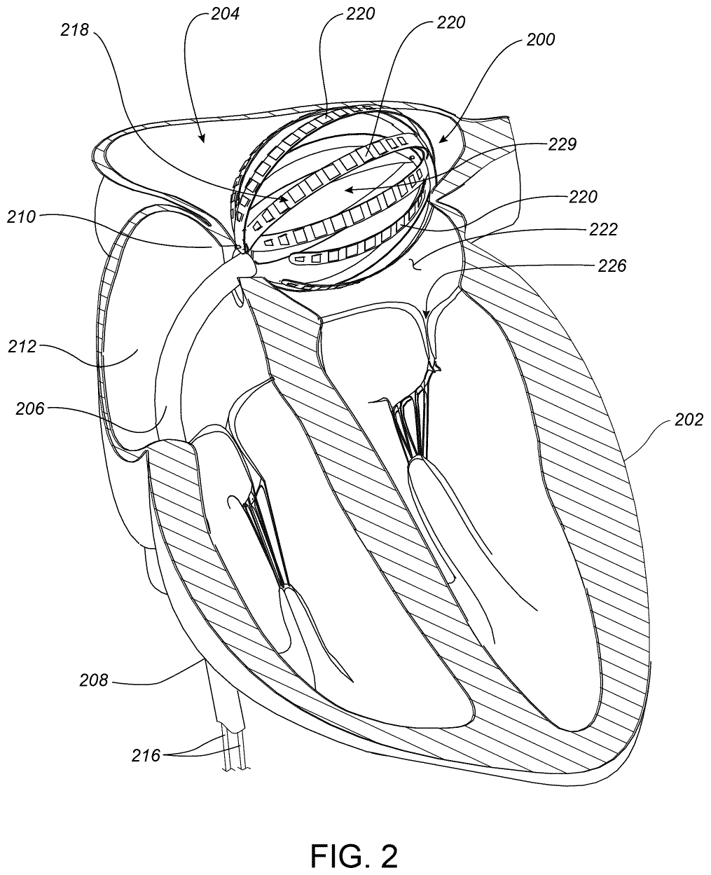

Various embodiments of transducer-based devices (e.g., forming part of catheters) are described herein in this disclosure. Some of the described devices are tissue ablation (e.g., PFA) devices that are percutaneously or intravascularly deployed. Some of the described devices are movable between a delivery or unexpanded configuration (e.g., FIG. 3A discussed below) in which a portion of the device is sized for passage through a bodily opening leading to a bodily cavity, and an expanded or deployed configuration (e.g., FIGS. 2 and 3B discussed below) in which the portion of the device has a size too large for passage through the bodily opening leading to the bodily cavity. An example of an expanded or deployed configuration, in some embodiments, is when the portion of the transducer-based device (e.g., catheter, or part thereof) is in its intended-deployed-operational state, which may be inside the bodily cavity when, e.g., performing an intended therapeutic or diagnostic procedure for a patient, or which may be outside the bodily cavity when, e.g., performing testing, quality control, or other evaluation of the device. Another example of the expanded or deployed configuration, in some embodiments, is when the portion of the transducer-based device (e.g., catheter, or part thereof) is being changed from the delivery configuration to the intended-deployed-operational state to a point where the portion of the device now has a size too large for passage through the bodily opening leading to the bodily cavity.

In some example embodiments, the described devices are part of a transducer-activation system capable of ablating tissue in a desired pattern within the bodily cavity using various techniques (e.g., via PFA, etc., according to various embodiments).

In some example embodiments, the devices are capable of sensing various cardiac functions (e.g., electrophysiological activity including intracardiac voltages which form the basis of recorded electrograms according to some embodiments). In some example embodiments, the devices are capable of providing stimulation (e.g., electrical stimulation) to tissue within the bodily cavity. Electrical stimulation may include pacing.

FIG. 2 is a representation of a transducer-based device 200 useful in investigating or treating a bodily organ, for example, a heart 202, according to at least one example embodiment.

Transducer-based device 200 can be percutaneously or intravascularly inserted into a portion of the heart 202, such as an intra-cardiac cavity like left atrium 204. In this example, the transducer-based device 200 is part of a catheter 206 inserted via the inferior vena cava 208 and penetrating through a bodily opening in transatrial septum 210 from right atrium 212. (In this regard, transducer-based devices or device systems described herein that include a catheter may also be referred to as catheters, catheter devices or catheter-based devices, in some embodiments). In other embodiments, other paths may be taken.

Catheter 206 includes an elongated flexible rod or shaft member appropriately sized to be delivered percutaneously or intravascularly. Various portions of catheter 206 may be steerable. Catheter 206 may include one or more lumens. The lumen(s) may carry one or more communications or power paths, or both. For example, the lumens(s) may carry one or more electrical conductors 216 (two shown). Electrical conductors 216 provide electrical connections to transducer-based device 200 that are accessible externally from a patient in which the transducer-based device 200 is inserted.

According to some embodiments, transducer-based device 200 includes a frame or structure 218 which assumes an unexpanded configuration for delivery to left atrium 204. Structure 218 is expanded (e.g., shown in a deployed or expanded configuration in FIG. 2) upon delivery to left atrium 204 to position a plurality of transducers 220 (three called out in FIG. 2) proximate the interior surface formed by tissue 222 of left atrium 204. In some embodiments, at least some of the transducers 220 are used to sense a physical characteristic of a fluid (e.g., blood) or tissue 222, or both, that may be used to determine a position or orientation (e.g., pose), or both, of a portion of a device 200 within, or with respect to left atrium 204. For example, transducers 220 may be used to determine a location of pulmonary vein ostia or a mitral valve 226, or both. In some embodiments, at least some of the transducers 220 may be used to selectively ablate portions of the tissue 222. In some embodiments, at least some of the transducers 220 are used to sense a physical characteristic of a fluid (e.g., blood) or tissue 222, or both, that may be used to determine contact or a degree of contact between a portion of a device 200 and a tissue surface (e.g., an internal tissue surface). In some embodiments, some of the transducers 220 may be used to ablate a pattern around the bodily openings, ports or pulmonary vein ostia, for instance to reduce or eliminate the occurrence of atrial fibrillation. In some embodiments, at least some of the transducers 220 are used to ablate cardiac tissue. In some embodiments, at least some of the transducers 220 are used to sense or sample intra-cardiac voltage data or sense or sample intra-cardiac electrogram data.

FIGS. 3A, 3B, and 3C (collectively, FIG. 3) include a transducer-based device system (e.g., a portion thereof shown schematically) that includes a catheter including a transducer-based device 300, according to some embodiments. Transducer-based device 300 includes a plurality of elongate members 304 (not all of the elongate members called out in each of FIGS. 3A, 3B, and 3C) and a plurality of transducers 306 (not all of the transducers called out in FIG. 3). FIG. 3C includes a representation of a portion of the transducer-based device 300 shown in FIG. 3B, but as viewed from a different viewing direction. It is noted that, for clarity of illustration, all the elongate members shown in FIGS. 3B and 3C are not represented in FIG. 3A. The plurality of transducers 306 are positionable within a bodily cavity. For example, in some embodiments, the transducers 306 are able to be positioned in a bodily cavity by movement into, within, or into and within the bodily cavity, with or without a change in a configuration of the plurality of transducers 306. In some embodiments, the plurality of transducers 306 are arrangeable into various spatial distributions including two-or three-dimensional distributions, grids or arrays of the transducers capable of mapping, ablating or stimulating an inside surface of a bodily cavity or lumen. As shown, for example, in FIG. 3A, the plurality of transducers 306 are arranged in a configuration that is receivable in a bodily cavity. In various ones of the FIG. 3, each of at least some of transducers 306 includes a respective electrode 315 (not all of the electrodes 315 called out in each of the FIG. 3). According to various embodiments, the input-output device system 120 is communicatively connected to each electrode in the spatial distribution of electrodes (e.g., the electrodes 315 coupled or attached to the structure 308) provided by the catheter.

According to some embodiments, the elongate members 304 may be arranged in a frame or structure 308 that is selectively movable between an unexpanded or delivery configuration (e.g., as shown in FIG. 3A) and an expanded or deployed configuration (e.g., as shown in FIGS. 3B, 3C) that may be used during a positioning of the elongate members 304 against a tissue surface of the bodily cavity or during a positioning of the elongate members 304 in the vicinity of the tissue surface. At least the expanded or deployed configuration shown in FIGS. 3B and 3C is an example of a spatial distribution of the transducers 306 (e.g., a three-dimensional spatial distribution). In some embodiments, structure 308 has a size in the unexpanded or delivery configuration suitable for delivery through a bodily opening (e.g., via catheter sheath 312 (shown in FIG. 3A but removed from FIGS. 3B and 3C for clarity)) to the bodily cavity. At least in a state in which the structure 308 is in the expanded or deployed configuration, the structure 308 may be considered to have two opposing poles 341a and 341b, marked by the intersection with axis 342 extending through the structure 308 as shown in FIGS. 3B and 3C according to some embodiments. In some embodiments, at least some of the plurality of transducers 306 are circumferentially arranged, e.g., in successive ring-like arrangements, about each of the poles 341a and 341b according to some embodiments. Two of such ring-like arrangements are illustrated, for example, as broken-line rings 343a and 343b in FIG. 3B and FIG. 3C, respectively. According to some embodiments, at least some of the plurality of transducers 306 are arranged in a plurality of groups of the transducers 306, the groups of transducers 306 arranged like lines of longitude (e.g., along respective elongate members 304) about the structure 308 between each of the poles 341a and 341b, according to some embodiments.

In some embodiments, structure 308 has a size in the expanded or deployed configuration too large for delivery through a bodily opening (e.g., via catheter sheath 312) to the bodily cavity. The elongate members 304 may form part of a flexible circuit structure (e.g., also known as a flexible printed circuit board (PCB)). The elongate members 304 may include a plurality of different material layers. Each of the elongate members 304 may include a plurality of different material layers. The structure 308 may include a shape memory material, for instance, Nitinol. The structure 308 can include a metallic material, for instance stainless steel, or non-metallic material, for instance polyimide, or both a metallic and non-metallic material by way of non-limiting example. The incorporation of a specific material into structure 308 may be motivated by various factors including the specific requirements of each of the unexpanded or delivery configuration and expanded or deployed configuration, the required position or orientation (e.g., pose), or both of structure 308 in the bodily cavity or the requirements for successful ablation of a desired pattern.

FIG. 4 is a schematic side elevation view of at least a portion of a transducer-based device 400 that includes a flexible circuit structure 401 that is employed to provide a plurality of transducers 406 (two called out) according to an example embodiment. In some embodiments, the flexible circuit structure 401 may form part of a structure (e.g., structure 308) that is selectively movable between a delivery configuration sized for percutaneous delivery and an expanded or deployed configuration sized too large for percutaneous delivery. In some embodiments, the flexible circuit structure 401 may be located on, or form at least part of, a structural component (e.g., elongate member 304) of a transducer-based device system.

The flexible circuit structure 401 can be formed by various techniques including flexible printed circuit techniques. In some embodiments, the flexible circuit structure 401 includes various layers including flexible layers 403a, 403b and 403c (e.g., collectively flexible layers 403). In some embodiments, each of flexible layers 403 includes an electrical insulator material (e.g., polyimide). One or more of the flexible layers 403 can include a different material than another of the flexible layers 403. In some embodiments, the flexible circuit structure 401 includes various electrically conductive layers 404a, 404b, and 404c (collectively electrically conductive layers 404) that are interleaved with the flexible layers 403. In some embodiments, each of the electrically conductive layers 404 is patterned to form various electrically conductive elements. For example, electrically conductive layer 404a is patterned to form a respective electrode 415 of each of the transducers 406. Electrodes 415 have respective electrode edges 415-1 that form a periphery of a respective electrically conductive surface portion associated with the respective electrode 415, the respective surface portion configured to be contactable with a tissue surface of a bodily cavity at least in some operating states, like a tissue ablation operating state. In some embodiments, the respective surface portions (e.g., one surface portion called out as 315-la in FIG. 3B) of the electrodes face outwardly from a central region (e.g., central region 229 in FIG. 2 or central region 329 in FIGS. 3B and 3C) within the three-dimensional distribution of the electrodes. It is noted that other electrodes employed in other embodiments may have electrode edges arranged to form different electrode shapes (for example, as shown by electrode edges 315-1 in FIG. 3B).

Electrically conductive layer 404b is patterned, in some embodiments, to form respective temperature sensors 408 for each of the transducers 406 as well as various leads 410a arranged to provide electrical energy to the temperature sensors 408. In some embodiments, each temperature sensor 408 includes a patterned resistive member 409 (two called out) having a predetermined electrical resistance. In some embodiments, each resistive member 409 includes a metal having relatively high electrical conductivity characteristics (e.g., copper). In some embodiments, electrically conductive layer 404c is patterned to provide portions of various leads 410b arranged to provide an electrical communication path to electrodes 415. In some embodiments, leads 410b are arranged to pass though vias in flexible layers 403a and 403b to connect with electrodes 415. Although FIG. 4 shows flexible layer 403c as being a bottom-most layer, some embodiments may include one or more additional layers underneath flexible layer 403c, such as one or more structural layers, such as a steel or composite layer. These one or more structural layers, in some embodiments, are part of the flexible circuit structure 401 and can be part of, e.g., elongate member 304. In some embodiments, the one or more structural layers may include at least one electrically conductive surface (e.g., a metallic surface) exposed to blood flow. In addition, although FIG. 4 shows only three flexible layers 403a-403c and only three electrically conductive layers 404a-404c, it should be noted that other numbers of flexible layers, other numbers of electrically conductive layers, or both, can be included.

In some embodiments, electrodes 415 are employed to selectively deliver ablation energy (e.g., PFA energy) to various tissue structures within a bodily cavity (e.g., an intra-cardiac cavity or chamber). The energy delivered to the tissue structures may be sufficient for ablating portions of the tissue structures.

Energy that is sufficient for tissue ablation may be dependent upon factors including transducer location, size, shape, relationship with respect to another transducer or a bodily cavity, material or lack thereof between transducers, et cetera.

In some embodiments, each electrode 415 is employed to sense or sample an electrical potential in the tissue proximate the electrode 415 typically at a different time than delivering PFA energy sufficient for tissue ablation. In some embodiments, each electrode 415 is employed to sense or sample intra-cardiac voltage data in the tissue proximate the electrode 415. In some embodiments, each electrode 415 is employed to sense or sample data in the tissue proximate the electrode 415 from which an electrogram may be derived. In some embodiments, each resistive member 409 is positioned adjacent a respective one of the electrodes 415. In some embodiments, each of the resistive members 409 is positioned in a stacked or layered array with a respective one of the electrodes 415 to form a respective one of the transducers 406. In some embodiments, leads 410a are arranged to allow for a sampling of electrical voltage in between resistive members 409. This arrangement allows for the electrical resistance of each resistive member 409 to be accurately measured. The ability to accurately measure the electrical resistance of each resistive member 409 may be motivated by various reasons including determining temperature values at locations at least proximate the resistive member 409 based at least on changes in the resistance caused by convective cooling effects (e.g., as provided by blood flow).

Referring to FIGS. 3A, 3B, and 3C, transducer-based device 300 can communicate with, receive power from or be controlled by a transducer-activation system 322 (e.g., via leads 317). In some embodiments, the transducer-activation system 322 represents one or more particular implementations of the system 100 illustrated in FIG. 1. In some embodiments, the transducer-based device 300 or the transducer-based device 200 may be considered part of the transducer-activation system 322 or 100. However, the transducer activation system 322 or 100, according to various embodiments, is not limited to including or interacting with either of the particular transducer-based devices 200, 300, and may include or interact with other one or more other types of transducer-based devices in some embodiments.

The transducer-activation device system 322 may include a controller 324 that includes a data processing device system 310 (e.g., which may be a particular implementation of data processing device system 110 from FIG. 1) and a memory device system 330 (e.g., which may be a particular implementation of the memory device system 130 from FIG. 1) that stores data and instructions that are executable by the data processing device system 310 to process information received from transducer-based device 300 or to control operation of transducer-based device 300, for example, activating various selected transducers 306 to ablate tissue (e.g., via PFA) and control a user interface (e.g., of input-output device system 320) according to various embodiments including at least those described below with respect to FIGS. 5 and 8. Controller 324 may include one or more controllers.

Transducer-activation device system 322 includes an input-output device system 320 (e.g., which may be a particular implementation of the input-output device system 120 from FIG. 1) communicatively connected to the data processing device system 310 (e.g., via controller 324 in some embodiments). Input-output device system 320 may include a sensing device system 325 configured to detect various characteristics including, but not limited to, at least one of tissue characteristics (e.g., electrical characteristics such as tissue impedance, tissue conductivity, tissue type, tissue thickness) and thermal characteristics. In this regard, the sensing device system 325 may include one, some, or all of the transducers 306 (or 406 of FIG. 4) of the transducer-based device 300, including the internal components of such transducers shown in FIG. 4, such as the electrodes 415 and temperature sensors 408.

Transducer-activation device system 322 may also include an energy source device system 340 (also referred to as power supply system in some embodiments) including one or more energy source devices connected to transducers 306. In this regard, although various ones of FIG. 3 show a communicative connection between the energy source device system 340 and the controller 324 (and its data processing device system 310), the energy source device system 340 may also be connected to the transducers 306 via a communicative connection that is independent of the communicative connection between the energy source device system 340 and the controller 324 (and its data processing device system 310). For example, the energy source device system 340 may receive control signals via the communicative connection with the controller 324 (and its data processing device system 310), and, in response to such control signals, deliver energy to, receive energy from, or both deliver energy to and receive energy from one or more of the transducers 306 via a communicative connection with such transducers 306 (e.g., via one or more communication lines through catheter body or shaft 314, elongated cable 316 or catheter sheath 312) that does not pass through the controller 324. In this regard, the energy source device system 340 may provide results of its delivering energy to, receiving energy from, or both delivering energy to and receiving energy from one or more of the transducers 306 to the controller 324 (and its data processing device system 310) via the communicative connection between the energy source device system 340 and the controller 324.

The energy source device system 340 may, for example, be connected to various selected transducers 306 or electrodes thereof to selectively provide energy in the form of electrical current or power (e.g., PFA energy) to cause ablation of tissue. The energy source device system 340 may, for example, selectively provide energy in the form of electrical current to various selected transducers 306, and such transducers 306 may measure a temperature characteristic, an electrical characteristic, or both at a respective location at least proximate each of the various transducers 306 utilizing energy provided by the energy source device system 340. The energy source device system 340 may include various electrical current sources or electrical power sources as energy source devices.

It is understood that input-output device system 320 may include various systems. In some embodiments, input-output device system 320 may include energy source device system 340, transducer-based device 300, or both energy source device system 340 and transducer-based device 300 by way of non-limiting example. Input-output device system 320 may include the memory device system 330 in some embodiments.

In other example embodiments, other structures besides those shown in FIGS. 2, 3A, 3B, 3C, and 4 may be employed to support or carry transducers of a transducer-based device such as a transducer-based catheter. For example, an elongated catheter member may be used to distribute the transducers in a linear or curvilinear array. Basket catheters or balloon catheters may be used to distribute the transducers in a two-dimensional or three-dimensional array.

According to some embodiments of the present invention, the system 100 (FIG. 1) includes some, or all, of the system 200 shown in FIG. 2, or vice versa. In some embodiments, the system 100 includes some, or all, of the system 300 in FIG. 3, or vice versa. In this regard, the system 200, the system 300, or each of the system 200 and the system 300 may be a particular implementation of the system 100, according to some embodiments. Some or all of the controller 324, energy source device system 340, or input-output device system 320 described with respect to FIG. 3 may also be implemented with the system 200 in FIG. 2, in some embodiments. Each of at least part of the transducer or electrode-based device system 400 in FIG. 4 may be part of the system 100, the system 200, or the system 300, according to various embodiments.

FIG. 5 includes a processing flow diagram, which may implement various embodiments of methods 500 by way of associated computer-executable instructions according to some example embodiments. In various example embodiments, a memory device system (e.g., memory device system 130 or 330, otherwise stated herein as “e.g., 130, 330”, such convention used for other references as well) is communicatively connected to a data processing device system (e.g., data processing device system 110, 310) and stores a program executable by the data processing device system to cause the data processing device system to execute various embodiments of methods 500 via interaction with at least, for example, a catheter including a transducer-based device (e.g., transducer-based device 200, 300, or 400, in some embodiments). In these various embodiments, the program may include instructions configured to perform, or cause to be performed, various embodiments of methods 500. In some embodiments, methods 500 may include a subset of the associated blocks or additional blocks than those shown in FIG. 5. In some embodiments, methods 500 may include a different sequence than those indicated between various ones of the associated blocks shown in FIG. 5. The features recited by any block are not intended to be exclusive, and the adopting of any recited features of any particular block in a particular embodiment does not prevent the inclusion of any other features, according to some embodiments, unless the features cannot work together (i.e., are mutually exclusive).

According to some embodiments, methods 500 may include block 502 associated with computer-executable instructions (e.g., reception instructions provided by a program) configured to cause a data processing device system (e.g., 110, 310) to receive a user-selection of a first group of electrodes in the spatial distribution of electrodes. For example, in FIG. 3C, a group of electrodes 315 is shown with a “diagonal-hatched” pattern in accordance with the FIG. 3C KEY to visually represent these particular electrodes 315 as being members of a user-selected first group 318A of electrodes 315.

In some embodiments associated with FIG. 3, the input-output device system 320 may include a user-activatable control that is responsive to a user action. Input-output device system 320 may include one or more user interfaces or input/output (I/O) devices, for example, one or more display device systems 332, speaker device systems 334, one or more keyboards, one or more mice (e.g., mouse 335), one or more joysticks, one or more track pads, one or more touch screens or other transducers to transfer information to, from, or both to and from a user, for example, a care provider such as a physician or technician. For example, output from a mapping process may be displayed on a display device system 332. Input-output device system 320 may include one or more user interfaces or input/output (I/O) devices, for example, one or more display device systems 332, speaker device systems 334, keyboards, mice, joysticks, track pads, touch screens or other transducers employed by a user to indicate a particular selection or series of selections of various graphical information. This graphical information may include various graphical elements whose user-selection thereof allows for the selection of particular ones of the transducers 306 or their associated electrodes 315. For example, in some embodiments, a graphical representation may include various selectable graphical elements, whose respective selection via particular user-input results in the selection of one or more corresponding transducers 306 or electrodes 315. Other form of user-selections may be employed additionally or alternatively in some embodiments. For example, in some embodiments, a user may input user-based information (e.g., via a keyboard of input-output device system 320) specifying or identifying a particular transducer or electrode set that is to be selected. Without limitation, various forms of user-based actions may be performed to enable a user-selection of the first group 318A of electrodes 315 as per block 502 according to various embodiments.

A user may select various patterns or arrangements of transducers 306/electrodes 315 from the spatial distribution of electrodes, according to various embodiments. For example, in some embodiments, the electrodes in the user-selected first group of electrodes are arranged in a ring-like arrangement, according to the spatial distribution. As best seen in FIG. 3C, this particular arrangement is exemplified by the ring-like arrangement of the first group 318A of user-selected electrodes 315, according to various embodiments. The particular arrangement pattern of the electrodes 315 in the user-selected first group 318A of electrodes 315 may be motivated for different reasons. For example, a ring-like arrangement may be chosen such that, when included in ablation activation (e.g., as per block 506, described below), a ring-like lesion may be formed. In some embodiments, a ring-like lesion may be employed to isolate tissue regions or anatomical regions circumscribed or otherwise surrounded by the ring-like lesion. For example, in the treatment of atrial fibrillation, ring-like lesions are typically formed around one or more pulmonary vein sets to isolate abnormal electrophysiological activity that may be associated with the pulmonary veins.

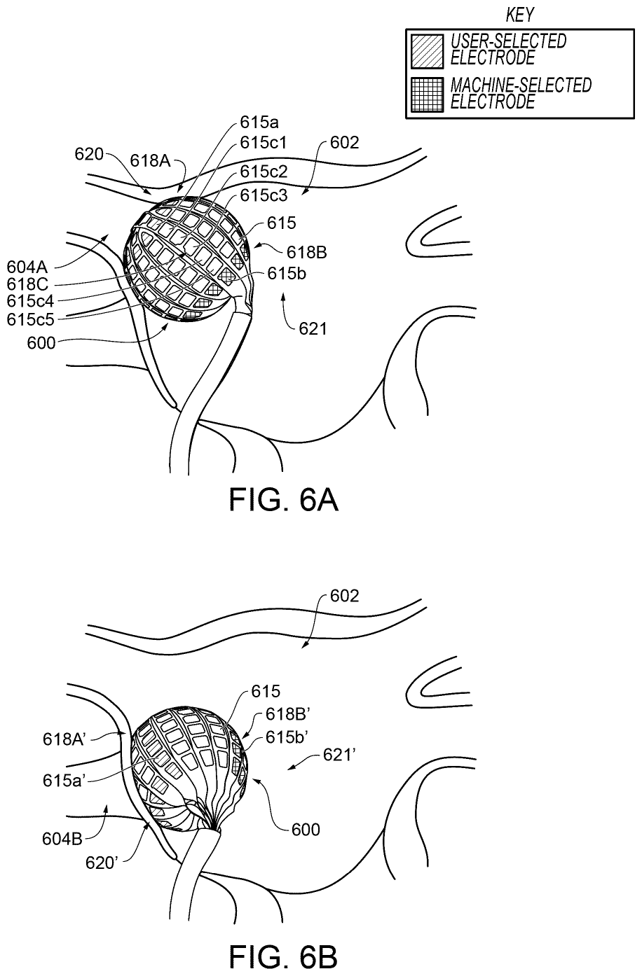

FIGS. 6A and 6B (collectively referred to as FIG. 6) show a catheter including a transducer-based device 600 including a spatial distribution of electrodes 615 (only one called out by reference numeral 615 in each of FIGS. 6A and 6B). Transducer-based device 600 is similar to, but not identical to, transducer-based device 300, and may employ the same or similar structures as and systems associated with transducer-based device 300. For example, transducer-based device 600 has some electrodes 615 that have different shapes than some of the electrodes 315 of transducer-based device 300. In FIG. 6, the catheter is manipulated to different locations within a cardiac chamber 602 according to various embodiments. In particular, in FIG. 6A, the transducer-based device 600 is positioned adjacent a first pulmonary vein 604A, and in FIG. 6B, the transducer-based device 600 is positioned adjacent a second pulmonary vein 604B. It is noted that different anatomical regions within a bodily cavity may require different orientations of the transducer-based device 600. These different orientations may occur for various reasons including particular physical constraints imposed by (i) the point of entry of the catheter, and (ii) the shape of the bodily cavity that may constrain various manipulations of the catheter within the bodily cavity.

Referring back to FIG. 3C, the user-selected first group 318A of electrodes 315 include, according to various embodiments, two ring-like arrangements of user-selected electrodes that are similar to ring-like arrangement 343b, in that they are each circumferentially or radially arranged about pole 341b or axis 342. Such an arrangement may be suitable for selection when the transducer-based device 300 is presented in proximity to a particular anatomical region (e.g., a pulmonary vein) with an orientation that is essentially parallel to axis 342, according to some embodiments. Such an orientation is exemplified in FIG. 6A by transducer-based device 600's orientation with respect to pulmonary vein 604A. In FIG. 6A, a group of electrodes (e.g., such as a group of electrodes 315) is shown with a “diagonal” hatch pattern in accordance with the FIG. 6A KEY to visually represent these particular electrodes as being members of a user-selected first group 618A of electrodes 615. In some embodiments, the respective surface portion (e.g., one instance of surface portion 315-1a is shown in FIG. 3B) of each of at least some electrodes in the user-selected first group (e.g., at least user-selected first group 618A in FIG. 6A or user-selected group 618A′ in FIG. 6B, for example) of electrodes (e.g., electrodes 315, 615) faces toward a particular region (e.g., region 620 in FIG. 6A or region 620′ in FIG. 6B, for example) of the tissue surface of the bodily cavity.

It is understood, however, that different electrodes or different arrangements of electrodes may be selected for the user-selected first group of electrodes according to various embodiments. The particular electrodes selected as per block 502 and their spatial arrangement may be dependent on various factors such as desired selected pattern of the electrodes and a particular orientation of the transducer-based device with respect to a particular anatomical region within the bodily cavity that is to be treated. For example, if a ring-like arrangement of electrodes 615 is desired to be selected as per block 502 to encircle pulmonary vein 604B in FIG. 6B, then the user-selected first group 618A′ of electrodes 615 would be selected from a lateral region of the transducer-based device 600 rather than about a pole region. In FIG. 6B, a group of electrodes 615 is shown with a “diagonal” hatch pattern in accordance with the FIG. 6B KEY to visually represent these particular electrodes 615 as being members of a user-selected first group 618A′ of electrodes 615.

It is noted that although a ring-like arrangement that is “two electrodes wide” is shown in FIG. 3C, other embodiments may employ a ring-like arrangement that is only “one electrode wide” (for example, as shown in each of FIGS. 6A and 6B) according to some embodiments, or may employ a ring-like arrangement that is “more than two electrodes wide” according to some embodiments. It is further noted that the first group of electrodes that are user-selected as per block 502 need not be limited to ring-like arrangements, and other arrangements of electrodes may be selected as desired according to various embodiments. For example, groups of electrodes arranged along a linear or curvilinear path may be selected according to block 502, or two-dimensional arrays of adjacent electrodes may be selected according to block 502.

According to various embodiments, electrodes that are selected as per block 502 are particular electrodes that are located proximate to, or in contact with, particular tissue regions that are desired to be ablated (e.g., particular tissue regions in which a lesion is desired to be formed). It is typically desired to have high degrees of electrode-to-tissue contact in thermal ablation procedures to reduce certain issues such the formation of thermally induced coagulum in the surrounding blood. PFA is typically not prone to these issues, but nonetheless, it is usually desired, in some embodiments, that relatively high levels of electrode-to-tissue contact be employed in PFA procedures to generate deeper, more efficacious lesions. According to some embodiments, the data processing device system 110, 310 may be configured at least to receive, via the input-output device system 120, 320, an electrode-to-tissue contact information set indicating a respective degree of electrode-to-tissue contact for each of at least some of the electrodes in the spatial distribution of electrodes. The data processing device system 110, 310 may then be configured to cause, via the input-output device system 120, 320, a visual presentation (e.g., via a user interface) of the electrode-to-tissue contact information set, in some embodiments. According to various embodiments, a user may review such visual presentation of the electrode-to-tissue contact information set to assist the user with selecting electrodes that have sufficient tissue contact for inclusion in the user-selected first group of electrodes. For example, in some embodiments, a particular electrode may be selected for inclusion in the user-selected first group of electrodes based at least on the electrode's proximity to a particular tissue region where it is desired to form a lesion in, and the electrode having a desired degree of electrode-to-tissue contact (e.g., according to the electrode-to-tissue contact information set). In some embodiments, the desired degree of electrode-to-tissue contact is at least some contact. In some embodiments, the desired degree of electrode-to-tissue contact is a maximum amount of possible contact between the electrode and the tissue during a typical procedure. In some embodiments, the desired degree of electrode-to-tissue contact corresponds to a desired contact force value, for example, as may be derived by the data processing device system 110, 310 from force sensor transducer elements, according to some embodiments. See, for example, U.S. Pat. No. 8,906,011 (Gelbart et al.), issued Dec. 9, 2014, describing such force sensors.

The electrode-to-tissue contact information set can take various forms. For example, in some example embodiments, the catheter may include transducers that sense characteristics (e.g., convective cooling, permittivity, force) that distinguish between fluid, such as a fluidic tissue (e.g., blood), and tissue forming an interior surface of the bodily cavity. Such sensed characteristics can allow a medical system to characterize contact, or a degree of contact, between a portion of the device and a tissue surface. These sensed characteristics can also be employed to map the cavity, for example, using positions of openings or ports into and out of the cavity to determine a position or orientation (e.g., pose), or both, of the portion of the device in the bodily cavity. In some example embodiments, the described systems employ a navigation system or electro-anatomical mapping system including electromagnetic-based systems and electropotential-based systems to determine a positioning of a portion of a device in a bodily cavity. These mapping systems may be employed to generate a model of the bodily cavity, the model further visually indicating a positioning of at least part of the catheter within the bodily cavity. In this regard, electrode-to-tissue contact information set may visually indicate, based on the model, which electrodes are in, are not in, or are in a relative degree of contact with a tissue surface. See, for example, U.S. Pat. No. 8,906,011 (Gelbart et al.), issued Dec. 9, 2014, which describes, among other things related to determining transducer-to-tissue contact, “sensing characteristics (e.g., convective cooling, permittivity, force) that distinguish between blood and non-blood tissue . . . [to] allow a medical system to map the cavity, for example using positions of openings or ports into and out of the cavity to determine a position and/or orientation (i.e., pose) of the medical device in the cavity.” (See U.S. Pat. No. 8,906,011 at column 9, lines 31-38).

With reference to U.S. Pat. No. 8,906,011, the convective cooling approach may be utilized to determine transducer-to-tissue contact and, consequently, the locations of openings or ports in a bodily cavity. Per U.S. Pat. No. 8,906,011, “One approach to determining the locations is to use the convective cooling of heated transducer elements by the blood. A slightly heated mesh of transducer elements positioned adjacent to the non-blood tissue that forms walls of the atrium and across the openings or ports of the atrium will be cooler at the areas which are spanning the openings or ports carrying blood flow.” (See U.S. Pat. No. 8,906,011 at column 9, lines 53-59). Also according to U.S. Pat. No. 8,906,011, “Another approach to determining the locations is to make use of the differing change in dielectric constant as a function of frequency between blood and non-blood tissue. A set of transducer elements positioned around the non-blood tissue that forms the interior surface of the atrium and across the openings or ports of the atrium monitor the ratio of the dielectric constant from 1 KHz to 100 KHz. Such can be used to determine which of those transducer elements are not proximate to non-blood tissue, which is indicative of the locations of openings or ports.” (See U.S. Pat. No. 8,906,011 at column 9, line 60 to column 10, line 2). Regarding force sensing, it is stated in U.S. Pat. No. 8,906,011 at column 10, lines 3-11 that, “Yet another approach to determining the locations is to sense a position of the non-blood tissue that forms the atrium walls using transducer elements that sense force (i.e., force sensors). A set of force sensing transducer elements positioned around the non-blood tissue that forms the interior surface of the atrium and across the openings or ports of the atrium can be used to determine which of the transducer elements are not in contact with the non-blood tissue, which is indicative of the locations of openings or ports.” Although U.S. Pat. No. 8,906,011 describes, among other things, determining whether or not transducer elements are in contact with tissue for purposes of building a map of a bodily cavity, the techniques for determining whether or not transducer elements are in contact with tissue can be used for other purposes, such as at least for assisting in the user or machine selections of electrodes per at least blocks 502 and 504 (block 504 being described in more detail below) in FIG. 5 of the present application, according to some embodiments. See also U.S. Pat. No. 11,918,303, issued Mar. 5, 2024 (Moisa), regarding catheter-based navigation systems for mapping a bodily cavity.

Referring back to FIG. 5, according to some embodiments, methods 500 may include block 504 associated with computer-executable instructions (e.g., instructions provided by a program) configured to cause a data processing device system (e.g., 110, 310) to cause, at least in response to the user-selection of the first group of electrodes as per block 502, a machine-selection of a second group of electrodes in the spatial distribution of electrodes. According to some embodiments, the machine-selected second group of electrodes does not include any electrode in the user-selected first group of electrodes. For example, in FIGS. 3B and 3C, a group of electrodes 315 is shown with a “cross-hatched” pattern in accordance with the FIGS. 3B and 3C KEYs to visually represent these particular electrodes 315 as being members of a machine-selected second group 318B of electrodes 315 selected in accordance with block 504. It is noted that none of the electrodes in user-selected first group 318A of electrodes are shown in FIG. 3B because of the particular perspective view of the transducer-based device 300 in such FIG. 3B.

In some embodiments, the machine-selection selects each electrode in the machine-selected second group of electrodes at least as a particular electrode in the spatial distribution of electrodes that is spaced, according to the spatial distribution, from an electrode in the user-selected first group of electrodes at least by a respective interposed electrode set in the spatial distribution of electrodes. For example, in FIGS. 3B and 3C, a group 318C of electrodes 315 is shown with a “dotted” pattern in accordance with the KEYs in FIGS. 3B and 3C to visually represent these particular electrodes 315 as being members of respective interposed electrode sets. In this regard, each respective interposed electrode set may be considered to be, in some embodiments, a set of one or more electrodes that is between an electrode in the user-selected first group of electrodes and an electrode in the machine-selected second group of electrodes. For instance, in some embodiments, if a first electrode, which is in the user-selected first group of electrodes, and a second electrode, which is in the machine-selected second group of electrodes, are deemed to be end points of a line segment, and, if a third electrode is intersected by that line segment, it may be determined that the third electrode is between the first and second electrodes. For another example, if the spatial distribution of electrodes (e.g., electrodes 315) is deemed to form a three-dimensional mathematical surface in which the electrodes are deemed to reside, and if the first electrode, which is in the user-selected first group of electrodes, and the second electrode, which is in the machine-selected second group of electrodes, are deemed to be end points of a geodesic that runs along the mathematical surface, and if a third electrode is intersected by that geodesic, it may be determined that the third electrode is between the first and second electrodes, according to some embodiments. Other embodiments may utilize other interpretations of “between”.

In some embodiments, the number of electrodes in a respective interposed electrode set may be utilized as a proxy to distance to ensure that the data processing device system 110, 310 is machine-selecting an electrode for the second group of electrodes that has a sufficient distance from the corresponding electrode in the user-selected first group of electrodes to provide the pseudo-monopolar ablation and its corresponding benefits described herein.

According to various embodiments, the electrodes in the machine-selected second group of electrodes may be located outwardly of or may surround the user-selected first group of electrodes. For example, in FIG. 3C, the machine-selected second group 318B of electrodes 315 are located outwardly of and surround (e.g., at least as considered from a perspective view toward pole 341b of structure 308 along axis 342) the ring-like arrangement of the user-selected first group 318A of electrodes 315. In some embodiments, the respective surface portion (e.g., one instance of surface portion 315-la is shown in FIG. 3B) of each of at least some electrodes in the machine-selected second group (e.g., machine-selected second group 618B in FIG. 6A or machine-selected second group 618B′ in FIG. 6B, for example) of electrodes (e.g., electrodes 315, 615) faces toward an innermost region (e.g., region 621 in FIG. 6A or region 621′ in FIG. 6B) within the volume of space defined by the bodily cavity (e.g., assuming the volume has boundaries that extend across the ports of the bodily cavity (e.g., PVs)). In some embodiments, the respective surface portion (e.g., one instance of surface portion 315-1a is shown in FIG. 3B) of each of at least some electrodes in the user-selected first group (e.g., at least user-selected first group 618A in FIG. 6A or user-selected group 618A′ in FIG. 6B, for example) of electrodes faces toward a particular region (e.g., region 620 in FIG. 6A or region 620′ in FIG. 6B, for example) of the tissue surface of the bodily cavity, and the respective surface portion of each of at least some electrodes in the machine-selected second group (e.g., machine-selected second group 618B in FIG. 6A or machine-selected second group 618B′ in FIG. 6B, for example) of electrodes not facing toward, in some embodiments, or faces away from, in some embodiments, the particular region of the bodily cavity.

According to various embodiments, each respective interposed electrode set does not include (a) any electrode in the user-selected first group of electrodes, (b) any electrode in the machine-selected second group of electrodes, or (a) and (b) (for example, as shown in FIGS. 3C, 6A and 6B).

According to some embodiments, each respective interposed electrode set includes a particular number of particular electrodes equal to or exceeding a threshold number of one or more electrodes. For example, as shown in FIG. 3C, the machine-selected second group 318B of electrodes includes an electrode 315b that is spaced, according to the spatial distribution (e.g., of the electrodes coupled or attached to the structure 308), from an electrode 315a in the user-selected first group 318A of electrodes by a respective interposed electrode set including a single electrode 315c. In this illustrated embodiment, the threshold number of one or more electrodes is “one”, such that, according to some embodiments, the electrode 315a in the user-selected first group of electrodes 318A is spaced from at least a closest electrode 315b in the machine-selected second group of electrodes 318B by the threshold number of one electrode in this example, the one electrode being electrode 315c making up its own interposed electrode set.

A respective interposed electrode set may include other numbers of electrodes in other embodiments. For example, In FIG. 6A, a machine-selected second group 618B of electrodes (e.g., selected according to block 504) includes an electrode 615b that is spaced, according to the spatial distribution, from an electrode 615a in the user-selected first group 618A of electrodes (e.g., selected according to block 502) by a respective interposed electrode set including five (5) electrodes 615c1, 615c2, 615c3, 615c4 and 615c5. According to some embodiments, the threshold number of electrodes associated with this FIG. 6A example may be another number of electrodes besides five (5) electrodes, and various factors (e.g., such as ensuring a sufficient or threshold distance between the user and machine-selected electrodes to ensure a pseudo-monopolar ablation as discussed herein) may be considered in spacing electrode 615a in the user-selected first group 618A of electrodes from electrode 615b in the machine-selected second group 618B of electrodes by a respective interposed electrode set. For example, in FIG. 6B, a machine-selected second group 618B′ of electrodes (e.g., selected according to block 504) includes an electrode 615b′ that is spaced, according to the spatial distribution, from an electrode 615a′ in the user-selected first group 618A′ of electrodes (e.g., selected according to block 502) by a respective interposed electrode set including three (3) electrodes. In some embodiments, the memory device system 130, 330 may store data indicative of the threshold number of electrodes. In some embodiments, the memory device system 130, 330 may store data indicative of the threshold distance.

In some embodiments, each respective interposed electrode set in the spatial distribution of electrodes may include at least two electrodes adjacent one another according to the spatial distribution (for example, as per various embodiments associated with FIGS. 6A and 6B). In some embodiments, each respective interposed electrode set in the spatial distribution of electrodes may include (i) an electrode that is adjacent a first electrode in the user-selected first group of electrodes according to the spatial distribution, and (ii) an electrode that is adjacent a second electrode in the machine-selected second group of electrodes according to the spatial distribution. For example, according to some embodiments associated FIG. 3C, electrode 315c is included in a respective interposed electrode set 318C and is an electrode that is adjacent a first electrode (i.e., electrode 315a) in the user-selected first group 318A of electrodes according to the spatial distribution, and is an electrode that is adjacent a second electrode (i.e., electrode 315b) in the machine-selected second group 318B of electrodes according to the spatial distribution. In some embodiments, the electrode that is adjacent the first electrode in the user-selected first group of electrodes according to the spatial distribution is other than the electrode that is adjacent the second electrode in the machine-selected second group of electrodes according to the spatial distribution. For example, according to some embodiments associated with FIG. 6A, electrode 615c1 is included in a respective interposed electrode set 618C, and is an electrode that is adjacent a first electrode 615a in the user-selected first group 618A of electrodes according to the spatial distribution, and electrode 615c5 is also included in the respective interposed electrode set, and is an electrode that is adjacent a second electrode 615b in the machine-selected second group 618B of electrodes according to the spatial distribution, electrode 6155 being other than electrode 615c1. Such embodiments may correspond to a respective interposed electrode set having two or more electrodes.

According to some embodiments, a first electrode in the machine-selected second group of electrodes is spaced, according to the spatial distribution, from a particular electrode in the user-selected first group of electrodes by the respective interposed electrode set and at least a second electrode in the machine-selected second group of electrodes, the second electrode adjacent the first electrode according to the spatial distribution. For example, in some embodiments associated with FIG. 3C, a first electrode 315d in the machine-selected second group 318B of electrodes, is spaced, according to the spatial distribution, from a particular electrode 315a in the user-selected first group 318A of electrodes by the respective interposed electrode set 318C (made up of electrode 315c) and at least a second electrode 315b in the machine-selected second group 318B of electrodes, the second electrode 315b being other than the first electrode 315d. In some embodiments, a particular electrode in the machine-selected second group of electrodes is spaced, according to the spatial distribution, from a first electrode in the user-selected first group of electrodes by the respective interposed electrode set and at least a second electrode in the user-selected first group of electrodes, the second electrode adjacent the first electrode according to the spatial distribution. For example, in some embodiments associated with FIG. 3C, a particular electrode 315b in the machine-selected second group 318B of electrodes is spaced, according to the spatial distribution, from a first electrode 315e in the user-selected first group 318A of electrodes by the respective interposed electrode set 318C (made up of electrode 315c) and at least a second electrode 315a in the user-selected first group 318A of electrodes, the second electrode 315a being other than the first electrode 315e.

Referring back to FIG. 5, according to some embodiments, methods 500 may include block 506 associated with computer-executable instructions (e.g., activation instructions provided by a program) configured to cause a data processing device system (e.g., 110, 310) to cause, via the input-output device system (e.g., 120, 320), particular activation of each electrode in the user-selected first group of electrodes and each electrode in the machine-selected second group of electrodes. According to various embodiments, the particular activation may be configured to cause bipolar pulsed field tissue ablation to occur while omitting at least any electrode of each respective interposed electrode set from undergoing any activation configured to cause bipolar pulsed field tissue ablation at least throughout the particular activation configured to cause bipolar pulsed field tissue ablation. For instance, the particular activation may be a bipolar pulsed field tissue ablation between the user-selected first group of electrodes and the machine-selected second group of electrodes, but not including the interposed electrode sets, in some embodiments. According to some embodiments, the particular activation may be a bipolar pulsed field tissue ablation between only the user-selected first group of electrodes and the machine-selected second group of electrodes, excluding any tissue-ablative activation of the interposed electrode sets while the particular activation occurs.

In some embodiments, the particular activation configured to cause bipolar pulsed field tissue ablation may include concurrent activation of all electrodes in the user-selected first group of electrodes and all electrodes in the machine-selected second group of electrodes to cause the bipolar pulsed field tissue ablation. According to various embodiments, the particular activation may be configured to cause bipolar pulsed field tissue ablation by causing an electric field sufficient to cause irreversible electroporation to be generated between the user-selected first group of electrodes and the machine-selected second group of electrodes, the machine-selected second group of electrodes spaced from the user-selected first group of electrodes by the respective interposed electrode sets.

The present inventor has discovered that this novel mode of bipolar pulsed field tissue ablation, which omits at least any electrode of each respective interposed electrode set from undergoing any activation configured to cause bipolar pulsed field tissue ablation at least throughout the particular activation configured to cause bipolar pulsed field tissue exhibits “pseudo-monopolar” behavior. The present inventor has determined that increased separation between the electrodes of the user-selected first group of electrodes (e.g., when driven with a first polarity in some embodiments) and the electrodes of the machine-selected second group of electrodes (e.g., when driven with a second polarity opposite the first polarity in some embodiments) allows, during the particular activation, the electric field surrounding a particular one of the activated electrodes to increasingly approximate that of a monopolar field.

This effect can be illustrated by using simplified math associated with two point sources. The voltage at a particular point as determined by two point current sources can be determined by using the principle of superposition. For a point-source-based model, the inverse square law applies for current density, and consequently by integration, the voltage decreases with the inverse of the radial distance from a first electrode of interest. However, the contribution from a second electrode (e.g., an opposite polarity electrode in a bipolar ablation) perpendicularly offset from the radial direction measurement also falls off with the inverse of the total distance to the point of interest. Due to the offset of the second electrode, it means that, by the Pythagorean theorem, the distance between the second electrode and a particular point of interest in the tissue located at a radial distance from the first electrode is given by the square root of the sum of the squared offset and the squared radial distance into the tissue from the first electrode. Consequently, as the size of the offset increases relative to the radial distance, then the contribution of the perpendicular offset to the total distance becomes dominating. In the limit (infinitely far away) the second electrode with the opposite polarity has no contribution to the superposition calculation and the electric field matches that of a purely monopolar scenario. Therefore, with sufficient separation between each electrode, the field locally around each electrode behaves as if the electrode was activated in a monopolar fashion, and, for the purpose of therapeutic application, may be termed pseudo-monopolar in manner very similar to monopolar ablation. However, a benefit of the inventive pseudo-monopolar approach can be that the external current path, such as by way of a conventional indifferent electrode, is not required and, therefore, cost savings, system efficiency, and system simplicity can be increased for at least this reason.

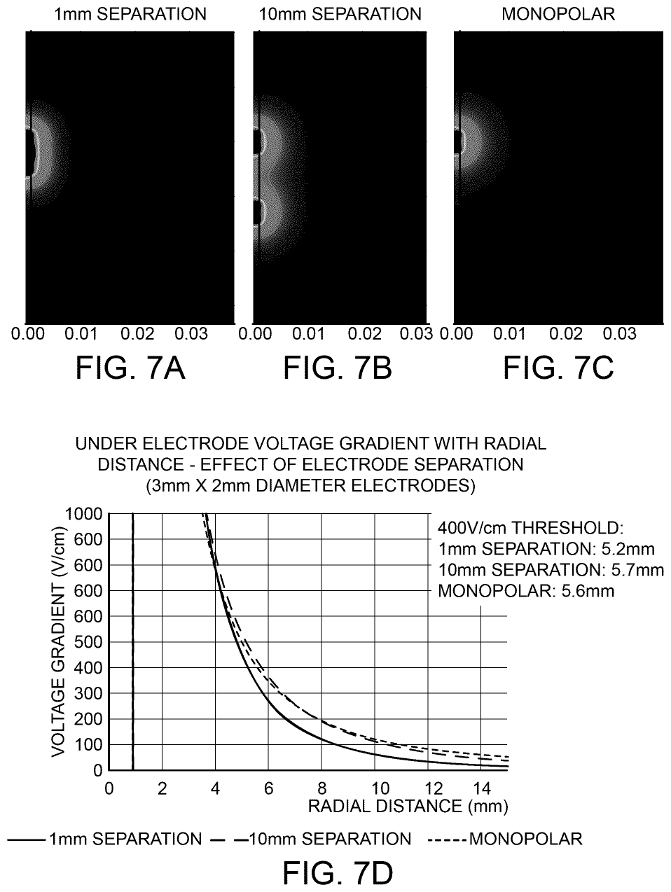

The inventive pseudo-monopolar effect is demonstrated in FIGS. 7A, 7B, and 7C, which model various electric field distributions for different electrode-to-electrode spacings (i.e., FIGS. 7A and 7B) and for monopolar PFA (FIG. 7C). The electrodes were each modeled with a diameter of 2 mm and a length of 3 mm. FIGS. 7A, 7B, and 7C were created by the present inventor using COMSOL 4.1 including the electric currents module (COMSOL, Inc., 100 District Avenue, Burlington, MA 01803, USA). In particular, FIG. 7A models the electric field generated by a bipolar pulsed field ablation employing two electrodes spaced 1 mm apart. FIG. 7B models the electric field generated by a bipolar pulsed field ablation employing two electrodes spaced 10 mm apart. FIG. 7C models the electric field generated by a monopolar pulsed field ablation employing a single internal electrode. The horizontal axes in FIGS. 7A, 7B, and 7C represent the distance from the electrode(s) in meters, and the lighter shades of gray in FIGS. 7A, 7B, and 7C represent stronger regions of the electric field compared to darker shades of gray.

In this regard, the electrode spacing in FIG. 7B is sufficiently large to produce two electric field regions that are each similar in form to the monopolar PFA case simulated in FIG. 7C. As compared with FIG. 7A, which simulates a typical bipolar PFA field, the electrode spacing associated with FIG. 7B is sufficiently large to produce the pseudo-monopolar effects described herein.

FIG. 7D is a graph showing how the voltage gradient for the same electrode voltage varies with the radial distance from an electrode (e.g., in the direction of lesion depth when the electrode is in contact with tissue), the graph generated from data employed to produce FIGS. 7A, 7B, and 7C. The three curves of the graph of FIG. 7D show a first curve reflecting the 1 mm electrode-to-electrode separation (e.g., bipolar PFA case), a second curve reflecting the 10 mm electrode-to-electrode separation (e.g., pseudo-monopolar PFA case), and a third curve reflecting the monopolar case. For cardiac applications, a voltage gradient on the order of 400-500V/cm may be utilized in some embodiments to irreversibly electroporate myocardium tissue. The graph in FIG. 7D indicates that a voltage gradient of 400V/cm can exist at a radial distance (or lesion depth in some embodiments) of 5.2 mm from the electrode for the bipolar PFA case, while the same voltage gradient of 400V/cm can exist at a radial distance (or lesion depth in some embodiments) of 5.7 mm from the electrode for the pseudo-monopolar PFA case. Advantageously, the same voltage gradient of 400V/cm can exist at a radial distance (or lesion depth in some embodiments) of 5.6 mm from the electrode for the monopolar PFA case. In this regard, the pseudo-monopolar PFA case is believed to provide approximately an 8% improvement over the bipolar PFA case, while almost equaling the performance of the monopolar case, according to some embodiments. Further, muscle stimulation is typically associated with field interactions at relatively larger radial distances from PFA-activated electrodes. FIG. 7D shows that the pseudo-monopolar curve crosses the monopolar curve. In this regard, FIG. 7D indicates that, in comparison with the monopolar case, the pseudo-monopolar curve is associated with smaller voltage gradients at increasing radial distances (e.g., greater than 14 mm), thereby indicating the possibility for a reduction in unwanted muscle stimulation being associated with the pseudo-monopolar PFA mode as compared with monopolar PFA, according to various embodiments. According to various embodiments, the threshold number of electrodes associated with block 504 may be associated with particular electrode-to-electrode spacing required to provide pseudo-monopolar pulsed field tissue ablation. In this regard, a threshold number of electrodes in each interposed electrode set may be utilized such that, according to their spacing in the spatial distribution of the electrodes, the interposed electrode sets provide the particular electrode-to-electrode spacing between the particular electrodes activated in a bipolar manner to cause pseudo-monopolar pulsed field tissue ablation.

In some embodiments, a threshold distance may be associated with a particular electrode-to-electrode spacing between two electrodes, which, when the two electrodes are activated in a bipolar manner to cause pulsed field tissue ablation, the two electrodes generate a particular voltage gradient region between the two electrodes that is insufficient to cause irreversible electroporation or a desired therapeutic effect. For instance, it can be understood from a least FIG. 7B, that, if the separation between the two electrodes is further increased, the voltage gradient region (e.g., at a midpoint) between the two electrodes will further decrease, such that the distance deemed sufficient to provide pseudo-monopolar behavior between the electrodes may be selected or determined to be at least a threshold distance at which tissue ablation does not occur (e.g., at a midpoint) between the two electrodes, according to some embodiments. The threshold distance may, in some embodiments, correspond, or map to, the spatial arrangement of a threshold number of electrodes in the interposed electrode set that is located between the two activated electrodes.

A potential advantage of various embodiments of the herein-described pseudo-monopolar ablation compared to traditional bipolar ablation is that the pseudo-monopolar field (e.g., FIG. 7B in some embodiments) with wider electrode separation may result in less heat deposition in the space between the electrodes since the zone of maximum bipolar field (e.g., FIG. 7A in some embodiments) has a marked decrease in voltage gradient with wide separation (e.g., comparing FIG. 7A to FIG. 7B in some embodiments). A potential advantage of various embodiments of the herein-described pseudo-monopolar ablation compared to traditional monopolar ablation can be less dependence of the near-electrode field on factors affecting the impedance of the external indifferent electrode in traditional monopolar ablation (subcutaneous fat, adhesion etc.), and, consequently, a voltage driven approach may become more practical as compared to a current driven approach.

The data processing device system 110, 310 may be configured to analyze other factors that have a bearing on which particular electrodes are machine-selected for inclusion in the machine-selected second group of electrodes per block 504. In this regard, a particular electrode may be machine-selected for inclusion in the machine-selected second group of electrodes based on other factors in addition to being spaced from a particular electrode in the user-selected first group of electrodes by a respective interposed electrode set, the respective interposed electrode set defining a particular number of electrodes (e.g., the threshold number of electrodes in some embodiments) sufficient to space the particular electrode in the machine-selected second group of electrodes from the particular electrode in the user-selected first group of electrodes to achieve the pseudo-monopolar effects described above. For example, in some embodiments, other factors such as electrode-to-tissue contact may be considered.

In some embodiments, the data processing device system 110, 310 may be configured at least by the program at least to receive, via the input-output device system 120, 320, an electrode-to-tissue contact information set indicating a respective degree of electrode-to-tissue contact for each of at least some of the electrodes in the spatial distribution of electrodes. Electrode-to-tissue contact information sets may be derived in various manners including those described above in this disclosure. In some embodiments, the data processing device system 110, 310 may be configured at least by the program to cause the machine-selection of the second group of electrodes in the spatial distribution of electrodes based at least on an analysis of the electrode-to-tissue contact information set, such that the machine-selection selects the machine-selected second group of electrodes in a manner that each of at least one electrode in the machine-selected second group of electrodes is an electrode in the spatial distribution of electrodes that experiences a particular degree of electrode-to-tissue contact that is less than a respective degree of electrode-to-tissue contact experienced by each electrode in the user-selected first group of electrodes. In some embodiments, the at least one electrode in the machine-selected second group of electrodes that is an electrode in the spatial distribution of electrodes that experiences a particular degree of electrode-to-tissue contact that is less than a respective degree of electrode-to-tissue contact experienced by each electrode in the user-selected first group of electrodes may include a first electrode in the spatial distribution of electrodes that experiences no electrode-to-tissue contact.

In some embodiments, the data processing device system 110, 310 may be configured at least by the program at least to receive, via the input-output device system 120, 320, an electrode-to-tissue contact information set indicating a respective degree of electrode-to-tissue contact for each of at least some of the electrodes in the spatial distribution of electrodes. Electrode-to-tissue contact information sets may be derived in various manners including those described above in this disclosure. In some embodiments, the data processing device system 110, 310 may be configured at least by the program to cause the machine-selection of the second group of electrodes in the spatial distribution of electrodes based at least on an analysis of the electrode-to-tissue contact information set, such that the machine-selection selects the machine-selected second group of electrodes in a manner that each electrode in the machine-selected second group of electrodes is an electrode in the spatial distribution of electrodes that experiences a particular degree of electrode-to-tissue contact that is less than a respective degree of electrode-to-tissue contact experienced by each electrode in the user-selected first group of electrodes. In some embodiments, the particular degree of electrode-to tissue contact may be no electrode-to-tissue contact.

In some embodiments, the data processing device system 110, 310 may be configured at least by the program at least to receive, via the input-output device system 120, 320, an electrode-to-tissue contact information set indicating a respective degree of electrode-to-tissue contact for each electrode in the spatial distribution of electrodes. Electrode-to-tissue contact information sets may be derived in various manners including those described above in this disclosure. In some embodiments, the data processing device system 110, 310 may be configured at least by the program at least to cause the machine-selection of the second group of electrodes in the spatial distribution of electrodes based at least on an analysis of the electrode-to-tissue contact information set, such that the machine-selection selects the machine-selected second group of electrodes in a manner that an average or median of the particular degree of electrode-to-tissue contacts experienced by the electrodes in the machine-selected second group of electrodes is less than a respective one of an average or median of electrode-to-tissue contacts experienced by the electrodes in the user-selected first group of electrodes.

Selecting, for inclusion in the machine-selected second group of electrodes, particular electrodes having associated degrees of electrode-to-tissue contact that are less than associated degrees of electrode-to-tissue contact experienced by various electrodes of the user-selected first group of user-selected electrodes may be motivated for different reasons. For example, according to some embodiments, the user-selection of the first group of electrodes may include the selection of particular electrodes adjacent to, proximate to, or in contact with particular tissue regions with a bodily cavity in which it is desired to directly treat (e.g., forming lesions to block electrophysiological activity) during the particular activation configured to cause bipolar pulsed field tissue ablation (e.g., block 506). Although, according to some embodiments, it may be desired treat tissue regions in proximity to the electrodes of the user-selected first group of electrodes, it may not be desired to treat tissue regions in proximity to the electrodes of the machine-selected second group of electrodes. However, according to some embodiments, the particular activation configured to cause bipolar pulsed field tissue ablation involves the transmission of energy configured to cause irreversible electroporation between the electrodes in the user-selected first group of electrodes and the electrodes in the machine-selected second group of electrodes (for example, to provide the pseudo-monopolar ablation benefits described above in this disclosure). This transmission of energy may thus cause particular tissue regions that are adjacent to, proximate to, or in contact with various electrodes of the machine-selected second group of electrodes to be treated (e.g., ablated) when there is no desire for such treatment to occur. Accordingly, in some embodiments, criteria for the selection of candidate electrodes for inclusion in the machine-selected second group of electrodes may include an assessment of degree of electrode-to-tissue contact associated with the candidate electrodes. In this regard, candidate electrodes associated with relatively lower degrees of electrode-to-tissue contact (i.e., as compared with the degrees of electrode-to-tissue contact associated with the electrodes of the user-selected first group of electrodes) may be preferred for inclusion in the machine-selected second group of electrodes, according to some embodiments. In some embodiments, candidate electrodes associated with no electrode-to-tissue contact may be preferred for inclusion in the machine-selected second group of electrodes, according to some embodiments.

It is noted, however, that information indicating an absence of electrode-to-tissue contact associated with a candidate electrode may, in some cases, be insufficient to determine whether tissue ablation will occur in the tissue regions adjacent to, proximate to, or in contact with various electrodes of the machine-selected second group of electrodes during that particular activation configured to cause bipolar pulsed field tissue ablation (e.g., as per block 506). In some embodiments, a candidate electrode may be selected for inclusion in the machine-selected second group of electrodes based on an information set indicating that the candidate electrode is separated or spaced from adjacent tissue by a distance that meets or exceeds a particular threshold distance. In some embodiments, the threshold distance is a particular distance reflecting a spacing (e.g., a minimum distance) between an electrode (if included in the machine-selected second group of electrodes) and surrounding tissue that would be sufficient to restrict or prevent the surrounding tissue from undergoing any substantial pulsed field ablation during the particular activation to cause bipolar pulsed field tissue ablation (e.g., as per block 506). According to some embodiments, the threshold distance may vary with one or more parameters of the pulsed field energy that is to be delivered by the particular activation configured to cause bipolar pulsed field tissue ablation (e.g., as per block 506). For instance, a particular threshold distance may correspond to a distance from an electrode in which the voltage gradient, which is governed by one or more parameters (e.g., such as at least pulse voltage) of the delivered pulsed field energy, is insufficient to cause irreversible electroporation. Information sets indicating electrode-to-tissue separation or spacing values may be derived in various manners including from information provided by a catheter navigation system according to some embodiments. See U.S. Pat. No. 11,918,303, issued Mar. 5, 2024 (Moisa), regarding catheter-based navigation systems. FIGS. 6A and 6B herein show cardiac applications with machine-selected second groups of electrodes 618B, 618B′ which are machine selected from electrodes that are essentially in the blood pool away from neighboring tissue according to various embodiments.

In this regard, in some embodiments, the tissue surface of a bodily cavity defines a volume of space within the bodily cavity, and, in some embodiments, the machine-selection (e.g., per block 504) may select each electrode in the machine-selected second group of electrodes at least as a particular electrode in the spatial distribution of electrodes that is closer to an innermost region of the volume of space defined by the bodily cavity than any electrode in the user-selected first group of electrodes for inclusion in the machine-selected second group of electrodes. For instance, with respect to FIGS. 6A and 6B and their respective machine-selected second groups 618B, 618B′ of electrodes, each electrode in the machine-selected second group of electrodes is closer to an innermost region of the volume of space of the illustrated cardiac cavity than any electrode in the corresponding user-selected first group 618A, 618A′, according to some embodiments.

As can be seen from FIGS. 6A and 6B, the electrodes of such second group of electrodes 618B, 618B′ that are closer to the innermost region of the volume within the cardiac cavity experience a particular degree of electrode-to-tissue contact (e.g., no electrode-to-tissue contact in this example) that is less than a respective degree of electrode-to-tissue contact experienced by each electrode in the respective user-selected first group 618A, 618A′ of electrodes. In this regard, in some embodiments, the data processing device system 110, 310 may be configured at least to receive, via the input-output device system 120, 320, an electrode-to-tissue contact information set indicating a respective degree of electrode-to-tissue contact for each of at least some of the electrodes in the spatial distribution of electrodes, and to cause the machine-selection of the second group of electrodes (e.g., second group 618B, 618B′) in the spatial distribution of electrodes based at least on an analysis of the electrode-to-tissue contact information set, such that the machine-selection (e.g., per block 504) selects the machine-selected second group of electrodes in a manner that each electrode in the machine-selected second group of electrodes is an electrode in the spatial distribution of electrodes that experiences a particular degree of electrode-to-tissue contact that is less than a respective degree of electrode-to-tissue contact experienced by each electrode in the user-selected first group of electrodes.

According to various embodiments, the number of electrodes forming at least one respective interposed electrode set may be adjusted to exceed the threshold number of one or more electrodes (e.g., as per block 504) to accommodate other factors such as those described above (e.g., the unwanted ablation of particular tissue regions). According to various embodiments, the at least one respective interposed electrode set can contain various electrodes, at least some of the various electrodes associated with different degrees of electrode-to-tissue contact. For example, in FIG. 6A, the respective interposed electrode set 618C includes electrodes 615c1, 615c2, 615c3, 615c4 and 615c5. Electrodes 615c1 and 615c2 are located in contact with tissue (not shown in this sectioned view) forming an entry region to a pulmonary vein, while electrodes 615c3, 615c4 and 615c5 are not in contact with tissue but are exposed to blood flow, according to various embodiments. In this regard, according to some embodiments, at least one electrode set of the respective interposed electrode sets in the spatial distribution of electrodes may include at least one electrode having no electrode-to-tissue contact (e.g., electrodes 615c3, 615c4 and 615c5). In some embodiments, the machine-selection (e.g., per block 504 in FIG. 5) selects each electrode in the machine-selected second group of electrodes at least as a particular electrode that is spaced from an associated electrode in the user-selected first group of electrodes at least by a respective interposed electrode set. In this regard, in some embodiments, at least one electrode set of the respective interposed electrode sets in the spatial distribution of electrodes may include at least one electrode having a lower degree of electrode-to-tissue contact than the associated electrode in the user-selected first group of electrodes. For example, according to some embodiments associated with FIG. 6A, electrode 615a is a tissue contacting electrode that forms part of the user-selected first group of electrodes 618A, while electrode 615c5 is a non-tissue contacting electrode that forms part of the respective interposed electrode set 618C. According to some embodiments, at least one electrode set of the respective interposed electrode sets in the spatial distribution of electrodes may include at least one electrode in the spatial distribution having a lower degree of electrode-to-tissue contact than any electrode in the user-selected first group of electrodes. For example, this may occur in the immediately preceding example when each electrode in the user-selected first group of electrodes 618A is a tissue contacting electrode. It is noted that, according to some embodiments, a particular electrode-to-tissue contact status of electrodes in a respective interposed electrode set is not a factor if activation of the electrodes in the respective interposed electrode set to cause bipolar pulsed field ablation is omitted.

Referring back to block 506 of FIG. 5, the particular activation may be configured to cause bipolar pulsed field tissue ablation by causing an electric field sufficient to cause irreversible electroporation to be generated between the user-selected first group of electrodes and the machine-selected second group of electrodes, the machine-selected second group of electrodes spaced from the user-selected first group of electrodes by the respective interposed electrode sets. In this regard, the particular activation of each electrode in the user-selected first group of electrodes and each electrode in the machine-selected second group of electrodes may take various forms according to some embodiments. For example, in some embodiments, the particular activation configured to cause bipolar pulsed field tissue ablation may be configured to cause a voltage difference between each electrode in the user-selected first group of electrodes and each electrode in the machine-selected second group of electrodes, the voltage difference having a magnitude sufficient to cause bipolar pulsed field tissue ablation. According to various embodiments, the voltage difference between each electrode in the user-selected first group of electrodes and each electrode in the machine-selected second group of electrodes may be established by delivering one or more high voltage pulse trains between each electrode in the user-selected first group of electrodes and each electrode in the machine-selected second group of electrodes. In some embodiments, each of the one or more high voltage pulse trains may include a plurality of biphasic pulses, the biphasic pulses configured to cause voltage polarity reversals. It is noted that, when a polarity reversal does occur, there still is a voltage difference between each electrode in the user-selected first group of electrodes and each electrode in the machine-selected second group of electrodes during the time of each active phase. It is further noted that, in some embodiments, the one or more high voltage pulse trains may be configured such that an inter-phase delay may exist between the phases or polarities of each biphasic pulse, and an inter-pulse delay may exist between successive biphasic pulses. According to various embodiments, each inter-phase delay and each inter-pulse delay are periods of effectively null generated voltage, and, consequently, there effectively is no voltage difference between each electrode in the user-selected first group of electrodes and each electrode in the machine-selected second group of electrodes during these delays, with the voltage difference being generated during the time of each active phase.

In some embodiments, the particular activation configured to cause bipolar pulsed field tissue ablation may include concurrent activation of all electrodes in the user-selected first group of electrodes and all electrodes in the machine-selected second group of electrodes to cause bipolar pulsed field tissue ablation. That is, in some embodiments, the voltage difference between each electrode in the user-selected first group of electrodes and each electrode in the machine-selected second group of electrodes may be established simultaneously or concurrently across all the electrodes in both the user-selected first group of electrodes and the machine-selected second group of electrodes. Such embodiments may have an advantage in at least some contexts of reducing the activation time of the particular activation configured to cause bipolar pulsed field tissue ablation. Concurrent activation may have the effect of making the cluster of electrodes made of the first and second groups of electrodes behave more as one large electrode, which can have a potential benefit in some contexts of achieving relatively greater lesion depth. In various embodiments associated with block 506, the particular activation is configured to cause bipolar pulsed field tissue ablation while omitting, at least throughout the particular activation configured to cause bipolar pulsed field tissue ablation, (1) generation of any particular voltage difference having a magnitude sufficient to cause bipolar pulsed field tissue ablation between each electrode in the user-selected first group of electrodes and each other electrode in the user-selected first group of electrodes, (2) generation of any particular voltage difference having a magnitude sufficient to cause bipolar pulsed field tissue ablation between each electrode in the machine-selected second group of electrodes and each other electrode in the machine-selected second group of electrodes, or (1) and (2). According to some of these embodiments, voltage differences having magnitudes sufficient to cause bipolar pulsed field tissue ablation are generated between the electrodes of the user-selected first group of electrodes and the electrodes of the machine-selected second group of electrodes while voltage differences having magnitudes sufficient to cause bipolar pulsed field tissue ablation are not generated between (a) the electrodes of the user-selected first group of electrodes, (b) the electrodes of the machine-selected second group of electrodes, or each of (a) and (b).

Various device architectures may be employed in this regard, according to some embodiments. For example, with reference to FIG. 3, energy source device system 340 (also referred to a power supply system 340) includes a first pole 344a (indicated with a “+” polarity) and a second pole 344b (indicated with a “−” polarity). According to some embodiments, the data processing device system 110, 310 may be configured at least by the program (e.g., per some embodiments of block 506) at least to cause, at least in response to the machine-selection of the second group of electrodes and via the input-output device system 120, 320, (i) each electrode in the user-selected first group of electrodes to be electrically connected to a first pole (e.g., 344a) of a power supply system, and (ii) each electrode in the machine-selected second group of electrodes to be electrically connected to a second pole (e.g., 344b) of the power supply system. According to various embodiments, the first pole (e.g., 344a) and the second pole (e.g., 344b) have opposite polarities (e.g., as indicated in FIG. 3) at least throughout the particular activation configured to cause bipolar pulsed field tissue ablation. The voltage signal providing the required voltage difference to cause the pulsed field bipolar tissue ablation of block 506 may be generated between the two poles (e.g., 344a, 344b) according to some embodiments. In some embodiments, the data processing device system 110, 310 may be configured at least by the program at least to cause the power supply system (e.g., 340) to generate a pulsed voltage signal set between the first pole and the second pole to cause the particular activation configured to cause bipolar pulsed field tissue ablation. In some embodiments, the pulsed voltage signal may be a biphasic pulsed voltage signal and the polarities of the poles (e.g., 344a, 344b) may change in accordance with the phase change associated with each biphasic pulse. Nonetheless, the opposite polarities of the poles will be maintained as the polarity of the first pole switches from positive to negative while the polarity of the second pole switches from negative to positive. In some embodiments, the data processing device system 110, 310 is configured at least by the program at least to cause (i) and (ii) at least throughout the particular activation configured to cause bipolar pulsed field tissue ablation, e.g., at least when inter-phase delays within pulses do not exist.

In various embodiments associated with block 506, the particular activation configured to cause bipolar pulsed field tissue ablation is configured to cause bipolar pulsed field tissue ablation to occur concurrently between each electrode in the user-selected first group of electrodes and all electrodes in the machine-selected second group of electrodes. In some of these various embodiments, a voltage difference having a magnitude sufficient to cause bipolar pulsed field tissue ablation to occur is established between each electrode in the user-selected first group of electrodes and all electrodes in the machine-selected second group of electrodes. In some embodiments, each electrode in the user-selected first group of electrodes may be activated sequentially in this manner. In some embodiments, the particular activation configured to cause bipolar pulsed field tissue ablation may include concurrent activation of all electrodes in the user-selected first group of electrodes and all electrodes in the machine-selected second group of electrodes to cause the bipolar pulsed field tissue ablation.

In various embodiments associated with block 506, the particular activation configured to cause bipolar pulsed field tissue ablation is configured to cause bipolar pulsed field tissue ablation to occur concurrently between each electrode in the machine-selected second group of electrodes and all electrodes in the user-selected first group of electrodes. In some embodiments, each electrode in the machine-selected second group of electrodes may be activated sequentially in this manner. In some embodiments, the particular activation configured to cause bipolar pulsed field tissue ablation may include concurrent activation of all electrodes in the user-selected first group of electrodes and all electrodes in the machine-selected second group of electrodes to cause the bipolar pulsed field tissue ablation.

In some embodiments, the machine-selection of the machine-selected second group of electrodes (e.g., block 504) may be configured to cause the machine-selected second group of electrodes to have the same as or at least within 10% of the number of electrodes in the user-selected first group of electrodes. In some embodiments, each electrode in the spatial distribution of electrodes includes a respective energy transmission surface configured to transmit tissue-ablative energy, and the machine-selection of the machine-selected second group of electrodes (e.g., block 504) may be configured to cause the respective energy transmission surfaces of the machine-selected second group of electrodes to have a total combined surface area that is the same as or at least within 10% of a total combined surface area of the respective energy transmission surfaces of the user-selected first group of electrodes.

Selecting the machine-selected second group of electrodes to have substantially an equivalent number of electrodes or to have a substantially equivalent combined surface area as the electrodes of the user-selected first group of electrodes may be motivated for different reasons. For example, during the particular activation configured to cause bipolar pulsed field tissue ablation (e.g., block 506), such ensuring of substantially combined surface areas between the user-selected first group of electrodes and the machine-selected second group of electrodes may be employed to help balance the voltage gradient and current flow, according to some embodiments.

FIG. 8 includes a processing flow diagram, which may implement various embodiments of methods 800 by way of associated computer-executable instructions according to some example embodiments. In various example embodiments, a memory device system (e.g., memory device system 130, 330) is communicatively connected to a data processing device system (e.g., data processing device system 110, 310) and stores a program executable by the data processing device system to cause the data processing device system to execute various embodiments of methods 800 via interaction with at least, for example, a catheter including a transducer-based device (e.g., transducer-based device 200, 300, or 400, in some embodiments). In these various embodiments, the program may include instructions configured to perform, or cause to be performed, various embodiments of methods 800. In some embodiments, methods 800 may include a subset of the associated blocks or additional blocks than those shown in FIG. 8. In some embodiments, methods 800 may include a different sequence than those indicated between various ones of the associated blocks shown in FIG. 8. The features recited by any block are not intended to be exclusive, and the adopting of any recited features of any particular block in a particular embodiment does not prevent the inclusion of any other features, according to some embodiments, unless the features cannot work together (i.e., are mutually exclusive).

According to some embodiments, methods 800 may include at least some aspects of methods 500 described above with respect to FIG. 5 (or vice versa), with some changes or additions noted below, according to various embodiments. In this regard, in some embodiments, methods 800 may include block 801 not present in FIG. 5. Block 801 may be associated with computer-executable instructions (e.g., reception instructions provided by a program) configured to cause a data processing device system (e.g., 110, 310) to receive, via the input-output device system (e.g., 120, 320), location information indicating a location of each of one or more portions of the catheter within the bodily cavity. According to some embodiments, such location information may be acquired from or derived from information from a catheter navigation system known in the art, such as that disclosed in U.S. Pat. No. 11,918,303, issued Mar. 5, 2024 (Moisa). According to some embodiments, such location information may be acquired from or derived from information from the electrodes (e.g., 315) themselves, for instance, from a tissue contact sensing function known in the art from the electrodes, such as that disclosed in U.S. Pat. No. 8,906,011 (Gelbart et al.), issued Dec. 9, 2014, discussed above.

In some embodiments, methods 800 may include block 802, which may be the same as block 502 described above with respect to FIG. 5. In some embodiments, methods 800 may include block 804a, which may be the same as block 504 described above with respect to FIG. 5, with an addition that the machine-selection of the second group of electrodes may be performed based at least on the user-selection of the first group of electrodes (e.g., per blocks 802 and 502) and based at least on an analysis of the received location information (e.g., received per block 801). Such analysis of the received location information may include a determination of which electrodes have a sufficient distance from electrodes in the user-selected first group of electrodes, a determination of which electrodes are experiencing no tissue contact or are experiencing a lesser-degree of tissue contact than electrodes in the user-selected first group of electrodes, a determination of which electrodes have a sufficient or threshold number of interposed electrodes between them and electrodes in the user-selected first group of electrodes, a determination of which electrodes are facing toward an innermost region within the volume of space defined by the bodily cavity or are located closer to an innermost region within the volume of space defined by the bodily cavity as compared to any electrode in the user-selected first group of electrodes, or a combination of some or all of these determinations, according to various embodiments, to facilitate the machine-selection of the second group of electrodes per block 804a.

In some embodiments, methods 800 may include block 806, which may be the same as block 506 described above with respect to FIG. 5.

While some of the embodiments disclosed above are described with examples of cardiac ablation, the same or similar embodiments may be used for ablating other bodily organs or any lumen or cavity into which the devices of the present invention may be introduced.

Subsets or combinations of various embodiments described above can provide further embodiments.

These and other changes can be made to the invention in light of the above-detailed description. In general, in the following claims, the terms used should not be construed to limit the invention to the specific embodiments disclosed in the specification and the claims but should be construed to include other transducer-based device systems including all medical treatment device systems and all medical diagnostic device systems in accordance with the claims. Accordingly, the invention is not limited by the disclosure, but instead its scope is to be determined entirely by the following claims.

What is claimed is:

1. A medical system comprising:
an input-output device system communicatively connectable to each electrode in a spatial distribution of electrodes provided by a catheter, each electrode in the spatial distribution of electrodes configured to be contactable with a tissue surface of a bodily cavity;
a memory device system storing a program; and
a data processing device system communicatively connected to the input-output device system and the memory device system, the data processing device system configured at least by the program at least to:
receive, via the input-output device system, a user-selection of a first group of electrodes in the spatial distribution of electrodes;
cause, at least in response to the user-selection of the first group of electrodes, a machine-selection of a second group of electrodes in the spatial distribution of electrodes, the machine-selected second group of electrodes not including any electrode in the user-selected first group of electrodes, and the machine-selection selecting each electrode in the machine-selected second group of electrodes at least as a particular electrode in the spatial distribution of electrodes that is spaced, according to the spatial distribution, from an electrode in the user-selected first group of electrodes at least by a respective interposed electrode set in the spatial distribution of electrodes, each respective interposed electrode set comprising a particular number of particular electrodes equal to or exceeding a threshold number of one or more electrodes, each respective interposed electrode set not including (a) any electrode in the user-selected first group of electrodes, and not including (b) any electrode in the machine-selected second group of electrodes; and
cause, via the input-output device system, particular activation of each electrode in the user-selected first group of electrodes and each electrode in the machine-selected second group of electrodes, the particular activation configured to cause bipolar pulsed field tissue ablation to occur while omitting at least any electrode of each respective interposed electrode set from undergoing any activation configured to cause bipolar pulsed field tissue ablation at least throughout the particular activation configured to cause bipolar pulsed field tissue ablation.

2. The medical system of claim 1, wherein the particular activation configured to cause bipolar pulsed field tissue ablation is configured to cause a voltage difference between each electrode in the user-selected first group of electrodes and each electrode in the machine-selected second group of electrodes, the voltage difference having a magnitude sufficient to cause bipolar pulsed field tissue ablation.

3. The medical system of claim 2, wherein the particular activation configured to cause bipolar pulsed field tissue ablation comprises concurrent activation of all electrodes in the user-selected first group of electrodes and all electrodes in the machine-selected second group of electrodes to cause bipolar pulsed field tissue ablation.

4. The medical system of claim 1, wherein the particular activation is configured to cause bipolar pulsed field tissue ablation while omitting, at least throughout the particular activation configured to cause bipolar pulsed field tissue ablation, (1) generation of any particular voltage difference having a magnitude sufficient to cause bipolar pulsed field tissue ablation between each electrode in the user-selected first group of electrodes and each other electrode in the user-selected first group of electrodes, (2) generation of any particular voltage difference having a magnitude sufficient to cause bipolar pulsed field tissue ablation between each electrode in the machine-selected second group of electrodes and each other electrode in the machine-selected second group of electrodes, or (1) and (2).

5. The medical system of claim 1, wherein the particular activation configured to cause bipolar pulsed field tissue ablation is configured to cause bipolar pulsed field tissue ablation to occur concurrently between each electrode in the user-selected first group of electrodes and all electrodes in the machine-selected second group of electrodes.

6. The medical system of claim 5, wherein the particular activation configured to cause bipolar pulsed field tissue ablation comprises concurrent activation of all electrodes in the user-selected first group of electrodes and all electrodes in the machine-selected second group of electrodes to cause the bipolar pulsed field tissue ablation.

7. The medical system of claim 1, wherein the particular activation configured to cause bipolar pulsed field tissue ablation is configured to cause bipolar pulsed field tissue ablation to occur concurrently between each electrode in the machine-selected second group of electrodes and all electrodes in the user-selected first group of electrodes.

8. The medical system of claim 7, wherein the particular activation configured to cause bipolar pulsed field tissue ablation comprises concurrent activation of all electrodes in the user-selected first group of electrodes and all electrodes in the machine-selected second group of electrodes to cause the bipolar pulsed field tissue ablation.

9. The medical system of claim 1, wherein the data processing device system is configured at least by the program at least to cause, at least in response to the machine-selection of the second group of electrodes and via the input-output device system, (i) each electrode in the user-selected first group of electrodes to be electrically connected to a first pole of a power supply system, and (ii) each electrode in the machine-selected second group of electrodes to be electrically connected to a second pole of the power supply system, the first pole and the second pole having opposite polarities at least throughout the particular activation configured to cause bipolar pulsed field tissue ablation.

10. The medical system of claim 9, wherein the data processing device system is configured at least by the program at least to cause the power supply system to generate a pulsed voltage signal set between the first pole and the second pole to cause the particular activation configured to cause bipolar pulsed field tissue ablation.

11. The medical system of claim 1, wherein the data processing device system is configured at least by the program at least to:
receive, via the input-output device system, an electrode-to-tissue contact information set indicating a respective degree of electrode-to-tissue contact for each of at least some of the electrodes in the spatial distribution of electrodes; and
cause the machine-selection of the second group of electrodes in the spatial distribution of electrodes based at least on an analysis of the electrode-to-tissue contact information set, such that the machine-selection selects the machine-selected second group of electrodes in a manner that each of at least one electrode in the machine-selected second group of electrodes is an electrode in the spatial distribution of electrodes that experiences a particular degree of electrode-to-tissue contact that is less than a respective degree of electrode-to-tissue contact experienced by each electrode in the user-selected first group of electrodes.

12. The medical system of claim 11, wherein the at least one electrode in the machine-selected second group of electrodes that is an electrode in the spatial distribution of electrodes that experiences a particular degree of electrode-to-tissue contact that is less than a respective degree of electrode-to-tissue contact experienced by each electrode in the user-selected first group of electrodes comprises a first electrode in the spatial distribution of electrodes that experiences no electrode-to-tissue contact.

13. The medical system of claim 1, wherein the data processing device system is configured at least by the program at least to:
receive, via the input-output device system, an electrode-to-tissue contact information set indicating a respective degree of electrode-to-tissue contact for each electrode in the spatial distribution of electrodes; and
cause the machine-selection of the second group of electrodes in the spatial distribution of electrodes based at least on an analysis of the electrode-to-tissue contact information set, such that the machine-selection selects the machine-selected second group of electrodes in a manner that an average or median of the respective degrees of electrode-to-tissue contacts experienced by the electrodes in the machine-selected second group of electrodes is less than a respective one of an average or median of degrees of electrode-to-tissue contacts experienced by the electrodes in the user-selected first group of electrodes.

14. The medical system of claim 1, wherein each respective interposed electrode set in the spatial distribution of electrodes comprises (i) an electrode that is adjacent a first electrode in the user-selected first group of electrodes according to the spatial distribution, and (ii) an electrode that is adjacent a second electrode in the machine-selected second group of electrodes according to the spatial distribution.

15. The medical system of claim 14, wherein the electrode that is adjacent the first electrode in the user-selected first group of electrodes according to the spatial distribution is other than the electrode that is adjacent the second electrode in the machine-selected second group of electrodes according to the spatial distribution.

16. The medical system of claim 1, wherein a first electrode in the machine-selected second group of electrodes is spaced, according to the spatial distribution, from a particular electrode in the user-selected first group of electrodes by the respective interposed electrode set and at least a second electrode in the machine-selected second group of electrodes, the second electrode adjacent the first electrode according to the spatial distribution.

17. The medical system of claim 1, wherein a particular electrode in the machine-selected second group of electrodes is spaced, according to the spatial distribution, from a first electrode in the user-selected first group of electrodes by the respective interposed electrode set and at least a second electrode in the user-selected first group of electrodes, the second electrode adjacent the first electrode according to the spatial distribution.

18. The medical system of claim 1, wherein at least one electrode set of the respective interposed electrode sets in the spatial distribution of electrodes comprises at least one electrode in the spatial distribution having a lower degree of electrode-to-tissue contact than any electrode in the user-selected first group of electrodes.

19. The medical system of claim 1, wherein at least one electrode set of the respective interposed electrode sets in the spatial distribution of electrodes comprises at least one electrode having a lower degree of electrode-to-tissue contact than the associated electrode in the user-selected first group of electrodes.

20. The medical system of claim 1, wherein the machine-selection of the machine-selected second group of electrodes is configured to cause the machine-selected second group of electrodes to have within 10% of the number of electrodes in the user-selected first group of electrodes.

21. The medical system of claim 1, wherein each electrode in the spatial distribution of electrodes comprises a respective energy transmission surface configured to transmit tissue-ablative energy, and wherein the machine-selection of the machine-selected second group of electrodes is configured to cause the respective energy transmission surfaces of the machine-selected second group of electrodes to have a total combined surface area that is within 10% of a total combined surface area of the respective energy transmission surfaces of the user-selected first group of electrodes.
